SEQUENCE LISTING (72 sequences)

SEQ ID NO: 1 - DNA, 20 bp, Artificial Sequence
  Fw primer of pair 1
gagacggcgaagaaggagat

SEQ ID NO: 2 - DNA, 20 bp, Artificial Sequence
  Rv primer of pair 1
ggatgtggaggatctggtgt

SEQ ID NO: 3 - DNA, 20 bp, Artificial Sequence
  Fw primer of pair 2
cggaatctaaggtcctcctg

SEQ ID NO: 4 - DNA, 20 bp, Artificial Sequence
  Rv primer of pair 2
gccttcttctttgcctgaga

SEQ ID NO: 5 - DNA, 23 bp, Artificial Sequence
  Fw primer of pair 3
gccggcccacgccgacacgctcg

SEQ ID NO: 6 - DNA, 20 bp, Artificial Sequence
  Rv primer of pair 3
gccttcttctttgcctgaga

SEQ ID NO: 7 - DNA, 23 bp, Artificial Sequence
  Fw primer of pair 4
gccggcccacgccgacacgctca

SEQ ID NO: 8 - DNA, 20 bp, Artificial Sequence
  Rv primer of pair 4
gccttcttctttgcctgaga

SEQ ID NO: 9 - DNA, 20 bp, Artificial Sequence
  Fw primer of pair 5
cggtgacgatactggtgatg

SEQ ID NO: 10 - DNA, 20 bp, Artificial Sequence
  Rv primer of pair 5
cttcaatggctgcaggacac

SEQ ID NO: 11 - DNA, 13155 bp, Mugil cephalus
tcagctcttcatctcctctcgtccaactgcatttgttggactcagtcaggctcatctcccttccatttctctttgtgcgagcagctgtgggagagtgtatctgtgtgtgtgcgtggccgtgtcaggggctcaaggctgcattcttcttccagcttcggaataaatcacaaagagcgccggtcctccacatgaagttcccacaacacacacacacacacacacacatacacacacacacacggagaggagaaattgaacattttaaaagaaggaaatctcaaaaacagtttgccacggaggcttgtgtacgtgtgcgcgcacatgaacgcacagtgtccttgcgaggtggcggcacgtcctggcgatgtgtgatgagccgagtagaaaggattattatgtccattgttatccgactgtgctcatcattcaacgcaaatatgacacagaagacaaaacagattcacacctgcgcggtctgcaaacttatgtttttgtgttcgatcgttgacctgaagctaaaagggggggcagaaaaccgatcaggtttcctactgtctgctatttactcttaaactgcaaataacgcagactcaactggaaaccaagtaacaaaaaacaagtggaaaaagtgtttttcttaaatcacagcatcctatcaggatgtaccgcgtgacgtgctgtggggtttttacacggtaaacacacacacgcacacacacacacaaagccaagcagagagacctcctacatcctcatgcacccttgaagcttttcacacgccccaaacattcaccgctgcccagcagaaaacgcctcacatgtcctctgtctgcgccctgagtgagtaaccggaccaaatacgtccgtataaaacgctggcgccggtggaagcctttgtctgcatgtgaacacagcgggggtggaggcaaaatcgttgcaaaagacacgaaagaagaaggaaataggtgaaaacacagtttggacgcagacaggctgaagatgatgacggtgacgatactggtgatgctgaagtttgcgatgataaagatggaggcggcctcggcacctggctttgagctgggcttcagacccggacgggagaccgttttggccaaacaaacctgcttcctggtgggacccggggccatggcgataccctccaacatgtccagcagcacccagtgcttgtgagtacgcaaacacacaaagcgaagcgtgtcctgcagccattgaaggtgtagaaacaaatccctcgaaggctagtgtagtatcaagttatgttgtggaattaaaggtaatactgtttaactgattcccattctgtttggcatttgtaccgaaaacaggatgagactttatgtatcattcttgacagcaccaccccttatcgacccctaaatgaagtgcattcagcatttatttgtcgttggttctttacttttctgtatccgctgtaagatttttttcataaaagtctacagcatgcagccattttgggagacagagaaaggtcaagactgcagcggaagagcccgagatatgctgactttttctcccgagccaagctccgaaaacctcttgatcccatttgttcaataatttatttcggcattccttcatccctttgtgaaacgctcacatctctcaaactcttccaccccggcttgttgcagaccctatgaagccgttcagggttatttctcagaactttagcgcgtttgctttgcagctacgggggtcgtggtcagaatatgatgaggagcgaatgggctttaatgtgtgaaatgtgaaccgtaacctacactcttaaaaatactacctcccattgcccgtctcatagcgatgcaggttaaggggacattcaatatttacaactaataatctcattttgtgtgcatgcataatcatttagactttcccctctaatcctccgttatctccccgtggatgaattttgcacttcacccctgtcagttccggttttattaaacattcattcataatgcatgtcaagcatgcgtgttcatcctcagcattttgcggtgcaagcacacctactgtcactgttggccatgcggtatcctcagcttaaagatgccgagtctcgtgagcaagagaaagttttctctctgaacaaaaactgtaacctcccaggttacagtattcctcttcgttaaattgctgcactgcacacaagggccatttgttgagccgctttgttgccactagttggacccctgatggctgcttttcagatccagttcgtttaacctgcacttcgatcactctgtgttgttcagctgttcagtttagagaaccggttcaggaaaaactagtagtttcactcctagctctacctagctacctagcctccgtgctaatatttgctcgcctggttttcgattctgctggtatgattagttattaactcctgtgtagaaagctagctggctattggctacggtctttgcaactttttggtccagacacaggtgacgtgaggcaatgtcccacttggccttcttcaccgattgctggttcgttcattcgttcgcgggggcagaagcttcaggagggaggtccagacggccctctccctgctcctctggaggaaccccccaggctaggcaggagatgtaatccaaccatcgtgtcctgggccaacctagaggcctccgtccggcgggacatgcctgaaacacctctaaccggaggtgtcctgaaggcatccttatcagatgcctgaaacacctcaactgactcctcttgatgtggaggagcagtggttctactctgagctcctcccgtatgtccaaattccttaccttatacctggtcaccaagcaaaggaaacttcgtccgtttgtatccgcgatcttgttctttcggtcattacccaaagttcatggccataggtgcaggttagaacgtagatcaaccagtaaattgagagctttgcctttcggcttagctctctcttcaccaaacagactggatgtcgacaggtggttgctgtttaatgccggcctttgcaatttttttcttttttttttgcatcatgttgcctgagtcatagcactgctgtccatgacttgcctttgtgaattaaatgcaaatgagtctcgcaatcggttagcactgatgcctcccagcgagaaggttctggttcgagtccagctcagacctttctgggtggagtttgtatgttctccctgtgtctgcgtgggttttctccaggtactccggtttcctcccacctccaaagacatgctcgttaggctaattggtaattccaaattgcccctagatgtgagtctgtgttagccctgtgatgggcttccaaccaccctagtgaggataagtggtagtagaagatgagatgtaatgagatcatcaaggaagtgcctaagcaatgacgattacacgggaacaccatgccaaaatatgacattccgaaaaaggctgaattcaaccccattcaaaaggaaaagataaatattcatggttttgcgtgacactacatatgaagcgcctacctgcagctgtctaactcagcccgaatactagaagcaggtggatacagcttgccaaagtatagtttttactccttgatgtagttacccttggatgagaaagcttagttttttatgttatgagattgacataggcgtcatatttagcatacagacaaaagagcgcatgtcaaggtatccgacttgtcatccaaacctgtgccagactctctgcgtgttatgtttcaaggccgtgcaagcgttgcagacctttagttcttgacacagtggcgcagagctgaagttgtagccatcaggggtccaacacctgatgttttggttgtttatgcatctggaaagaatctcaaggcctcgaagagaaaggggagggttgtcataaagattagattcacctgaagcgtttgcaggaacatgcatatgatcgtgcatgaggacagctttgctatgcaagtcataaacgtactttgtcgtagaagtagtgactattcccaacatatgcatgtgatcaactgcgtgtgttctcatccgttctgctcaaaggtcaaggctaaggaacagcagggatatttacaagtatgcagcagctgatggccatttcaaaagtgattcacaacagccagatgcaaaaaagctcggggtgttgtacagttcctaaacacgatagacaccgtttgtgtcctgcatgaaaagaaaatgcgaggaactagcctcctttattagtctccactcatgattgtccgctgtaaatttgtaacgtgtttctgcatccacagggaactcaagcagacacagatcagagtgattcccaagggtgccctcaccagcctgcagcacctcaggagactgtaagctttagtgaaattgacaaaaagaaatatctgtgtcaatatctgtaccggcatttgcatcctttttttcttttctttttcgtcttctatccactacttccttccttccaactttaggaacatactgcagaatgacgaactgcagagtatcagtgatctcgccttcgtcggcctccctcgtctcacttacatgtgagtgtgtggatttctaggagtcgctacatgcagagtattttatccaacatgcttgcgtgtttagtctttaaaatggaacaaaacaataggatcgcccaaatagctgggaagtaagctcggcaaacatggtggagacaaaatacagcctttagactttatttgtcccagaagagagatttgtttccacttgactgtacatgtattaatcacaacataagatcggacaagacaagagagtgctcaaatcaagaacagcttatggcattggacattaaatgaggtggacatagatggcagacacaggggagaagaaagaagtctgttatttagggtggatagggctgcaggtattaagctctttaccgtgcaagctcgtcagaatttaatcatccggtatctgcaacctgatgattttgtgtgataggaattaattttgtcccactggtgactgaaagcatggtggaggaacgtctaacagtataatgtttaaacatactacgtgtcttgtctttgtctgccccagcttaatatcagaaaatgttgctttagaaagaattggagcttccgccttctctgatctccctgaactcgttgagatgtaagtataactgacagagtaagggggtaaggagagtgttgagaccctttaatctcaagaccagagatataaactaatgagtgcattattgactgtcctcctttcagaaccataacaaagtcaagaaacctgcatttcatccactcagacgccttcagaaacctcatgaaactaaggtttatgtgagtactcagtcaacatatatagtttgtgtttttcagactggttatgagtccgatatgggccatgggtgacaggcctacttctgaatggccagagacatcattttttaattagcttggatccccggaagagaaatcaaatgtcacacttaatacagaggtttaaatacacatatatatatatattatttttttcccccactgaattacttacccactatttcagataagccacgctgagtaaaattttcaacgtaaccattcgtaaagggaagtgaactaatgtccttaaaagatgttatcataactgtcactgttgtatttgtcccattttatgtaaactgggacactgggacacgggagtctgtgctgatggacatctcttacttctaaacaagggcacagtagcacaggaaaacttatgttgtaatttgttggtaatttgtttaagataaggtttatttttttctacattcaatgaaaaaagcttataaaaataaaaaaaacaagctagtgaaatgtcttgatcactatcgggagggatttcttattgaatgaagtgcagatatttattgtaccaagaggatgactttaatggccccagttaaatttaaacgtgtcagcaaacacctgcaaaatttctaaggctcagctgatgttagcatcctgaatatgatatgatttatcctgctaattttactgcttatcttaaaccagtgctcaaaggattttgtttagtttttttcaaactgaaagcttgtatatcctaacgatcaacgttttacaggctgagaaacagacactaaatgtcttcattttgttttatctaagtgaataagtgaatgtaattacatttgaggtttctttcctcccgtctcgtcacaggaccatctcctacaccggcctgaaaatctttcccaacttttcccaggtccactccacctacaatgaatttcagctgtgagtctcactactagatagaaacggactggaaacacatgcagagatacgcacccacacgctgctcttgtgtacgcttctgcacactaaaatatgactagaggaaatcacaccaacatatagagtatacaacactgcagtcacgatctatgcaatgagaagcgctaacagattactcaagctatctaccttctagcttggtcttagtatctaaagcacacatactgttcatctgtcattttttttccacttgaaaattctactgttttctccacagtatttcttatttattgtgtgtttttgatcttttgacactcagtcacctgcaggagaacagcgacttaaagaaagtccccgccaatgccttcagaggcctctgcactaaaactatcaagaagatgtaagggcttcagcgcattttttttttttttttttattatttcttatttttttcttggtttgttttttcttgtttttcagaaatgcaccacaaccctcacacacatctgagtgggcatttcatgcacggtgatgaatgtttcctgaaatgctctctgccctcttcgctggctgcactttctcctgcttttctcctgcttttctcaaattctgcagaatcactcttacttgtgttgatcagcttttattttcctatccttttaagctgcagatattgaatcctgcttaaactgacttgctgaatcacactctgtgttagactgttagagagcgatcgcaaacatttactaagtttccttttaaatacatattattattgtagacttcatcagtttaacagaagagatatttcctgttgaagacttttatcaactgttggagatactaagatgtaaacacattcactatttatcaaaatgttcagaaacattctgtaaatgttctcagtaaaaacactgagctgctcttcgattactactgacatataaataatagcctatttcacactgcttttttattatttacttctactctagtgagattaatatttttattttaaaccagctgcaacattaaaatgcagtctattacattcagtaaaaataataatacaattttacagtatagctactgtatgataacatttgacaagacattaacagattctgtcacgtttgttgaaaatatttccttcacttttacatgaagatctaaagctaagctatggctagcagcaggttagcctagctcgttttttttttttaactatctaaaaaagcactgtttgtatttttttcactcacaagttgttgttttgcttgtcagttaatgagtcttggtggtaggtaggtttttctaagctgtggactaagtcagacgagctgtacttccttcatttttgcttaaatatatagctaagctatagctagcagcaggttagcctagctcgttttttttttttttttttaactatctaaaaaagtagtttgtatttttttcactcacaaaaacaatacagtgttttaatgggtgagttttagagatgaattaaaaaaaaacaaaaaaaaaaaaaaaacaacagatattggcaaagctacaccagaggagcatcaatctcatataggctaactctgatatgtctcatatgatgtatgtcaaattccacggtaaagctttgaatttatagctttgaatttataattttttacatgttaaagtttgaacgtgtctcacgaattccatgttaatcagctaaaagggaggaatttccaaatttcttagcgtgtttcacttgaaatccgttgcatttctgtcgagtcggtgaaagagaacaaaagaggtgcaaaggctaaaattagcgtcgagagatcagaacagaagtttgctgctggaaatgttgtgcgttggagccacgcgactgtaacttggcgtctcttgctattgacagatggctcaccagaaatggcatcaaggaggtggcaagtgacgctttcaatggaacaaagatgcaaagactgtgagtcacaggcagagacacaattacaagctgaagatatataaaaaaaaaataaaaaagtccgcattcaaaatatcttgccctctttcttttccccttagatttctgaaaggcaatcggcagcttactcacatgagtcccaacgccttcgcgggttccagtgagttggaagtgctgtaagcatgacatcttgatgtaactcatgcccacatgattcaccctgctcatcatcacttgacttaggatgatgcaggagaaacccagaatacatatggggaggctaaataaaaccttgtagaaatacaaaatatacagagagcatttttattttttttttcattgctgtgatgtttttgtcttcgttccctgcaattacttggcaatcatatcatttttttttttctggtgtatttttgtgtggattagtttgtcctgtgagaaataagtttttatcaactgatcttaggtcaaatctaaatagaaaaacagataacaagtagttgataaaaaaataaaaataaaaatcttgctgctgtgtttctccttgctcccacagtgacatctcccaaacagcgctcagctctctgccaggctccatcctcgggggactcaagagactgatcgcagagtcagccaagaacctgaagaagcttcctcctcctcggttcttcaccaaactgcaccaggccaacctgacgtactcgtctcactgctgcgccttccagaacatgcagagaaacaagtgcgcgtctcctgctcgactgttatagctcatggtttacctggaaagaatgggaaaaaaaaaaaaacacaaatacataggttgttcacgagtactgtacttgtgtgtgtgctagatcaaggaacaaccacaacctgtgcgctataccagaggcgaagaatgagcccttcttctacagggcgcactgctccacctccatcacctgcagcccggtgccggacgactttaacccgtgtgaggacatcatgtccgctgtccccttacgcatcctcatctggatcatctctatcctcgcgctactggggaacgcagtggttgtccttgtattgttgagtatgaaatgcttctcttgcattttatttcaatcatactgtgcatgcaattgtgtatataatcttgtatttccttgtgtggtagtagaaatagtagtaaatgtgcctttttgaatgctcttttctttcctccattgtcaggcagcagatcgaagctcaccgttccccgtttcctcatgtgccacttggccttcgctgacctctgcatgggaatctacctggcagtcatagcagccgtagacatgttatatcgtggcaagtactacaaccactccatagactggcaggagggcttgggctgcaaggctgccggcttcttctcggtgtgtacgcgagagctgacttcatggctcacataacaggcagcagctttggtcgcctctgagcggtaaacgaacgttgtctgtgtgtcacgtgcaggtgtttgccagcgagctgtcggtgttcacgttgacggcgatcaccgtggagcgctggcacaccatcacgcatgccctgcgcctggaccgtaagcttcgcctgagacacgcctgcatcatcatgacggccggctggatcttcgccgcggcggccgcgcttctgcccacggtcggggtcagcagctacagcaaagtgggtgttaaatttagctagtgcatgttcctctcagaattggctcaggtccttttagtttgggatgcaaaatggagaatagaatagaatagaataatctatcagttacacactgaatcaaacaactgactgacacatgaaaggaaatgtgaatgaaatgtgattaaaaagtataaaacaatctcaataattagaatcccaattagcctaatcaaatatcttgtgtgagaagaaaaggcataatctcactactatgaaactggaaccttattatatattattattgttccaatggccagaaggtcttttttaacaaagcttatagtatagtatatattatagtatatagtatagttatgatgcactgagcatcatccctcagcatcatcctccattttctcttctctggcaccgagctcctgtcctctaatctcttataattacttttctactgctaacccctcttcttcttgtggggatctttggcctggagtgctgcatatctggcttatggagctccaaactacatctgctgtcttggtctcatcagcctgcccagtctttttggccccctgcagttatccactcctacctggatgaacaattctccaacctgcccatgattcctcttacatgctcaaactgtcacataacgtcatcagctatatattgtattattagattctacatgtttgtgtacgataatatgaattgaattgaaattgaagatggagattgtaggattacacagatagtgtgcaaactggaatgagggaagcctgcactggctgtttcagagtgtagcttctgtgttgcaccttggaactttttggatagtatttgacaatatgtgaacaaatctgaactgcaataaacgcacatttaaatgtgctctttctgtttttactcaactgcaacatgtttagttgaggtcaacgtactgctcgtttcagcttcttagcaggaagacttttagcccatgttatggagtggaaatggagtgaactaggcttactctgtttttgtatatgtgtctgttcgtgcaggtgagcatctgcttgcccatggatgtggaggatctggtgtcccaggtctatgtagtgtccctgctcctcctcaacatcctggccttcctctgtgtgtgcggctgctacctcagcatctacctgacattccgcaatccctcttcggtgccggcccacgccgacacgcgcatggctcaacgcatggccgtcctcatcgtcaccgacttcatctgcatggccccgatctccttcttcgccgtctcggccgcgctcaagacccccctcatcaccgtctcggaatctaaggtcctcctggtcctgttctaccccattaactcgtgcgccaaccccttcctgtacgccttcttcacccgcaccttccggcgggacttctttttcctggcggctcgcttcggcctgtttaagactcgggcgcagatttaccggacagagacctcttcctgtcagcagccagcatggacctcttcgaggagcagccgcgtgacaatgtactccttggccaacaccttgagcctggacgcgtgcgtagactcccagtcgtccaagtcatggtagaccaaatcgcatgagtggatttgtgcttcgtttcgcttcttgaagatgtttcacctgttatccaggaggcttctaggactagatgagaaacaagagtgctgactttcatccaaatatccacagaagacgcacattcccctggtttgtgaacagaatgattctattttatctccacacaaaagctattttctctcaggcaaagaagaaggctagctctgaaggacataaggcagtctttaatgcacgaacacctgtgttcaacatgcagctctcgaggtgatttattttcaaggctgcatgcagcaggggtgtacaaacaccttttgatgtatctgccgagtttttgggcacgaaaagaaatcatacctgcataccgctgtcttatccctatttctgctttaattccacgtggacattgcagagtttcaagctcagtgtatctgacctcagatggacagcaacacagcgaagacacagtgtccagtgtcctccttaaaccttgtgatccacgggcaaaaacactgtagcaatccaagaaaagaaagctacggaagaactgcggatagtttgatcggatctaggtcaaacagctgtcccagatacccctgtgtttgttttaacttgattggagtgccagatgggcgaggttctccgcaacgttatttagaagtaaaactagaaagagtagacgatgtagtgaactggtgatttcattcttatgcaagaaacgccatccatctggtgtcccgagataacaaaacaaaccactctgaaaacagtttaacaaatgctttgggctagggacatcagcatatgcacgacatgaacagtctgactggatgatcttcattcagattaatacagcagagacctgctgattgataatctcctcatacactcagtctcctgtcctccaaatccactaacactttagcatctctttttcatttaattctattttttattttattttttatggaggacctacagttatctctgaagactaattgtgcatgtagtttgtaaacagcgcctcactttttatatctatgtgttgtgtacaagatgtatctttggtatgtttttttttatgtcattttataaagctgcttatttcatcctttccacctgagtgcattttgcgtgacgaatgcatgatgagttgtggcgaaaggaaaaactcaaacacacacgtgtgcataattgagccgcctttatctagcaccgctgtccttcggagcatccaaagctttcaaggacaaagtgaccacacacaaaaatttgctccatgtatcaaggtaggttggtgtgggaggagagactacacagctgctctctctgcttattacaggaggatggatattgcagctagataagacacacacacacacatactccagcagcaacccagagtttcaaggctgcagctgcaaggaggaatctgagttcatctcattgatatggatacactgcctcctgcaagccaacgccgaacgtgtgggcgcatttatgcaatttaaccaccaggtgatgctagtgatgccgtattaattgcgatatctgctctgaaggacagc

SEQ ID NO: 12 - protein, 80 aa, Mugil cephalus
YLTFRNPSSVPAHADTRMAQRMAVLIVTDFICMAPISFFAVSAALKTPLITVSESKVLLVLFYPINSCANPFLYAFFTRT

SEQ ID NO: 13 - DNA, 2118 bp, Mugil cephalus
atgatgacggtgacgatactggtgatgctgaagtttgcgatgataaagatggaggcggcctcggcacctggctttgagctgggcttcagacccggacgggagaccgttttggccaaacaaacctgcttcctggtgggacccggggccatggcgataccctccaacatgtccagcagcacccagtgcttggaactcaagcagacacagatcagagtgattcccaagggtgccctcaccagcctgcagcacctcaggagactgaacatactgcagaatgacgaactgcagagtatcagtgatctcgccttcgtcggcctccctcgtctcacttacatcttaatatcagaaaatgttgctttagaaagaattggagcttccgccttctctgatctccctgaactcgttgagataaccataacaaagtcaagaaacctgcatttcatccactcagacgccttcagaaacctcatgaaactaaggtttatgaccatctcctacaccggcctgaaaatctttcccaacttttcccaggtccactccacctacaatgaatttcagcttcacctgcaggagaacagcgacttaaagaaagtccccgccaatgccttcagaggcctctgcactaaaactatcaagaagatatggctcaccagaaatggcatcaaggaggtggcaagtgacgctttcaatggaacaaagatgcaaagactatttctgaaaggcaatcggcagcttactcacatgagtcccaacgccttcgcgggttccagtgagttggaagtgcttgacatctcccaaacagcgctcagctctctgccaggctccatcctcgggggactcaagagactgatcgcagagtcagccaagaacctgaagaagcttcctcctcctcggttcttcaccaaactgcaccaggccaacctgacgtactcgtctcactgctgcgccttccagaacatgcagagaaacaaatcaaggaacaaccacaacctgtgcgctataccagaggcgaagaatgagcccttcttctacagggcgcactgctccacctccatcacctgcagcccggtgccggacgactttaacccgtgtgaggacatcatgtccgctgtccccttacgcatcctcatctggatcatctctatcctcgcgctactggggaacgcagtggttgtccttgtattgttgagcagcagatcgaagctcaccgttccccgtttcctcatgtgccacttggccttcgctgacctctgcatgggaatctacctggcagtcatagcagccgtagacatgttatatcgtggcaagtactacaaccactccatagactggcaggagggcttgggctgcaaggctgccggcttcttctcggtgtttgccagcgagctgtcggtgttcacgttgacggcgatcaccgtggagcgctggcacaccatcacgcatgccctgcgcctggaccgtaagcttcgcctgagacacgcctgcatcatcatgacggccggctggatcttcgccgcggcggccgcgcttctgcccacggtcggggtcagcagctacagcaaagtgagcatctgcttgcccatggatgtggaggatctggtgtcccaggtctatgtagtgtccctgctcctcctcaacatcctggccttcctctgtgtgtgcggctgctacctcagcatctacctgaccttccgcaatccctcttcggtgccggcccacgccgacacgcgcgtggctcaacgcatggccgtcctcatcttcaccgacttcatctgcatggccccgatctccttcttcgccgtctcggccgcgctcaagacccccctcatcaccgtctcggaatctaaggtcctcctggtcctgttctaccccatcaactcgtgcgccaaccccttcctgtacgccttcttcacccgcaccttccggcgggacttctttttcctggcggctcgcttcggcctgtttaagactcgggcgcagatttaccggacagagacctcttcctgtcagcagccagcatggacctcttcgaggagcagccgcgtggcaatgtactccttggccaacaccttgagcctggacgcgtgcgtagactcccagtcgtccaagtcatggtag

SEQ ID NO: 14 - protein, 705 aa, Mugil cephalus
MMTVTILVMLKFAMIKMEAASAPGFELGFRPGRETVLAKQTCFLVGPGAMAIPSNMSSSTQCLELKQTQIRVIPKGALTSLQHLRRLNILQNDELQSISDLAFVGLPRLTYILISENVALERIGASAFSDLPELVEITITKSRNLHFIHSDAFRNLMKLRFMTISYTGLKIFPNFSQVHSTYNEFQLHLQENSDLKKVPANAFRGLCTKTIKKIWLTRNGIKEVASDAFNGTKMQRLFLKGNRQLTHMSPNAFAGSSELEVLDISQTALSSLPGSILGGLKRLIAESAKNLKKLPPPRFFTKLHQANLTYSSHCCAFQNMQRNKSRNNHNLCAIPEAKNEPFFYRAHCSTSITCSPVPDDFNPCEDIMSAVPLRILIWIISILALLGNAVVVLVLLSSRSKLTVPRFLMCHLAFADLCMGIYLAVIAAVDMLYRGKYYNHSIDWQEGLGCKAAGFFSVFASELSVFTLTAITVERWHTITHALRLDRKLRLRHACIIMTAGWIFAAAAALLPTVGVSSYSKVSICLPMDVEDLVSQVYVVSLLLLNILAFLCVCGCYLSIYLTFRNPSSVPAHADTRVAQRMAVLIFTDFICMAPISFFAVSAALKTPLITVSESKVLLVLFYPINSCANPFLYAFFTRTFRRDFFFLAARFGLFKTRAQIYRTETSSCQQPAWTSSRSSRVAMYSLANTLSLDACVDSQSSKSW

SEQ ID NO: 15 - DNA, 2118 bp, Mugil cephalus
atgatgacggtgacgatactggtgatgctgaagtttgcgatgataaagatggaggcggcctcggcacctggctttgagctgggcttcagacccggacgggagaccgttttggccaaacaaacctgcttcctggtgggacccggggccacggcgataccctccaacatgtccagcagcacccagtgcttggaactcaagcagacacagatcagagtgattcccaagggtgccctcaccagcctgcagcacctcaggagactgaacatactgcagaatgacgaactgcagagtatcagtgatctcgccttcgtcggcctccctcgtctcacttacatcttaatatcagaaaatgttgctttagaaagaattggagcttccgccttctctgatctccctgaactcgttgagataaccataacaaagtcaagaaacctgcatttcatccactcagacgccttcagaaacctcatgaaactaaggtttatgaccatctcctacaccggcctgaaaatctttcccaacttttcccaggtccactccacctacaatgaatttcagcttcacctgcaggagaacagcgacttaaagaaagtccccgccaatgccttcagaggcctctgcactaaaactatcaagaagatatggctcaccagaaatggcatcaaggaggtggcaagtgacgctttcaatggaacaaagatgcaaagactatttctgaaaggcaatcggcagcttactcacatgagtcccaacgccttcgcgggttccagtgagttggaagtgcttgacatctcccaaacagcgctcagctctctgccaggctccatcctcgggggactcaagagactgatcgcagagtcggccaagaacctgaagaaacttcctcctcctcggttcttcaccaaactgcaccaggccaacctgacgtactcgtctcactgctgcgccttccagaacatgcagagaaacaaatcaaggaacaaccacaacctgtgcgctataccagaggcgaagaatgagcccttcttctacagggcgcactgctccacctccatcacctgcagcccggtgccggacgactttaacccgtgtgaggacatcatgtccgctgtccccttacgcatcctcatctggatcatctctatcctcgcgctactggggaacgcagtggttgtccttgtattgttgagcagcagatcgaagctcaccgttccccgtttcctcatgtgccacttggccttcgccgacctctgcatgggaatctacctggcggtcatagcagccgtagacatgttatatcgtggcaagtactacaaccactccatagactggcaggagggcttgggctgcaaggctgccggcttcttctcggtgtttgccagcgagctgtcggtgttcacgttgacggcgatcaccgtggagcgctggcacaccatcacgcatgccctgcgcctggaccgtaagcttcgcctgagacacgcctgcatcatcatgacggccggctggatcttcgccgcggcggccgcgcttctgcccacggtcggggtcagcagctacagcaaagtgagcatctgcttgcccatggatgtggaggatctggtgtcccaggtctatgtagtgtccctgctcctcctcaacatcctggccttcctctgtgtgtgcggctgctacctcagcatctacctgaccttccgcaatccctcttcggtgccggcccacgccgacacgcgcgtggctcaacgcatggccgtcctcatcttcaccgacttcatctgcatggccccgatctccttcttcgccgtctcggccgcgctcaagacccccctcatcaccgtctcggaatctaaggtcctcctggtcctgttctaccccatcaactcgtgcgccaaccccttcctgtacgccttcttcacccgcaccttccggcgggacttctttttcctggcggctcgcttcggcctgtttaagactcgggcgcagatttaccggacagagacctcttcctgtcagcagccagcatggacctcttcgaggagcagccgcgtggcaatgtactccttggccaacaccttgagcctggacgcgtgcgtagactcccagtcgtccaagtcatggtag

SEQ ID NO: 16 - protein, 705 aa, Mugil cephalus
MMTVTILVMLKFAMIKMEAASAPGFELGFRPGRETVLAKQTCFLVGPGATAIPSNMSSSTQCLELKQTQIRVIPKGALTSLQHLRRLNILQNDELQSISDLAFVGLPRLTYILISENVALERIGASAFSDLPELVEITITKSRNLHFIHSDAFRNLMKLRFMTISYTGLKIFPNFSQVHSTYNEFQLHLQENSDLKKVPANAFRGLCTKTIKKIWLTRNGIKEVASDAFNGTKMQRLFLKGNRQLTHMSPNAFAGSSELEVLDISQTALSSLPGSILGGLKRLIAESAKNLKKLPPPRFFTKLHQANLTYSSHCCAFQNMQRNKSRNNHNLCAIPEAKNEPFFYRAHCSTSITCSPVPDDFNPCEDIMSAVPLRILIWIISILALLGNAVVVLVLLSSRSKLTVPRFLMCHLAFADLCMGIYLAVIAAVDMLYRGKYYNHSIDWQEGLGCKAAGFFSVFASELSVFTLTAITVERWHTITHALRLDRKLRLRHACIIMTAGWIFAAAAALLPTVGVSSYSKVSICLPMDVEDLVSQVYVVSLLLLNILAFLCVCGCYLSIYLTFRNPSSVPAHADTRVAQRMAVLIFTDFICMAPISFFAVSAALKTPLITVSESKVLLVLFYPINSCANPFLYAFFTRTFRRDFFFLAARFGLFKTRAQIYRTETSSCQQPAWTSSRSSRVAMYSLANTLSLDACVDSQSSKSW

SEQ ID NO: 17 - DNA, 2118 bp, Mugil cephalus
atgatgacggtgacgatactggtgatgctgaagtttgcgatgataaagatggaggcggcctcggcacctggctttgagctgggcttcagacccggacgggagaccgttttggccaaacaaacctgcttcctggtgggacccggggccatggcgataccctccaacatgtccagcagcacccagtgcttggaactcaagcagacacagatcagagtgattcccaagggtgccctcaccagcctgcagcacctcaggagactgaacatactgcagaatgacgaactgcagagtatcagtgatctcgccttcgtcggcctccctcgtctcacttacatcttaatatcagaaaatgttgctttagaaagaattggagcttccgccttctctgatctccctgaactcgttgagataaccataacaaagtcaagaaacctgcatttcatccactcagacgccttcagaaacctcatgaaactaaggtttatgaccatctcctacaccggcctgaaaatctttcccaacttttcccaggtccactccacctacaatgaatttcagcttcacctgcaggagaacagcgacttaaagaaagtccccgccaatgccttcagaggcctctgcactaaaactatcaagaagatatggctcaccagaaatggcatcaaggaggtggcaagtgacgctttcaatggaacaaagatgcaaagactatttctgaaaggcaatcggcagcttactcacatgagtcccaacgccttcgcgggttccagtgagttggaagtgcttgacatctcccaaacagcgctcagctctctgccaggctccatcctcgggggactcaagagactgatcgcagagtcagccaagaacctgaagaagcttcctcctcctcggttcttcaccaaactgcaccaggccaacctgacgtactcgtctcactgctgcgccttccagaacatgcagagaaacaaatcaaggaacaaccacaacctgtgcgctataccagaggcgaagaatgagcccttcttctacagggcgcactgctccacctccatcacctgcagcccggtgccggacgactttaacccgtgtgaggacatcatgtccgctgtccccttacgcatcctcatctggatcatctctatcctcgcgctactggggaacgcagtggttgtccttgtattgttgagcagcagatcgaagctcaccgttccccgtttcctcatgtgccacttggccttcgctgacctctgcatgggaatctacctggcagtcatagcagccgtagacatgttatatcgtggcaagtactacaaccactccatagactggcaggagggcttgggctgcaaggctgccggcttcttctcggtgtttgccagcgagctgtcggtgttcacgttgacggcgatcaccgtggagcgctggcacaccatcacgcatgccctgcgcctggaccgtaagcttcgcctgagacacgcctgcatcatcatgacggccggctggatcttcgccgcggcggccgcgcttctgcccacggtcggggtcagcagctacagcaaagtgagcatctgcttgcccatggatgtggaggatctggtgtcccaggtctatgtagtgtccctgctcctcctcaacatcctggccttcctctgtgtgtgcggctgctacctcagcatctacctgaccttccgcaatccctcttcggtgccggcccacgccgacacgcgcgtggctcaacgcatggccgtcctcatcttcaccgacttcatctgcatggccccgatctccttcttcgccgtctcggccgcgctcaagacccccctcatcaccgtctcggaatctaaggtcctcctggtcctgttctaccccatcaactcgtgcgccaaccccttcctgtacgccttcttcacccgcaccttccggcgggacttctttttcctggcggctcgcttcggcctgtttaagactcgggcgcagatttaccggacagagacctcttcctgtcagcagccagcatggacctcttcgaggagcagccgcgtgacaatgtactccttggccaacaccttgagcctggacgcgtgcgtagactcccagtcgtccaagtcatggtag

SEQ ID NO: 18 - protein, 705 aa, Mugil cephalus
MMTVTILVMLKFAMIKMEAASAPGFELGFRPGRETVLAKQTCFLVGPGAMAIPSNMSSSTQCLELKQTQIRVIPKGALTSLQHLRRLNILQNDELQSISDLAFVGLPRLTYILISENVALERIGASAFSDLPELVEITITKSRNLHFIHSDAFRNLMKLRFMTISYTGLKIFPNFSQVHSTYNEFQLHLQENSDLKKVPANAFRGLCTKTIKKIWLTRNGIKEVASDAFNGTKMQRLFLKGNRQLTHMSPNAFAGSSELEVLDISQTALSSLPGSILGGLKRLIAESAKNLKKLPPPRFFTKLHQANLTYSSHCCAFQNMQRNKSRNNHNLCAIPEAKNEPFFYRAHCSTSITCSPVPDDFNPCEDIMSAVPLRILIWIISILALLGNAVVVLVLLSSRSKLTVPRFLMCHLAFADLCMGIYLAVIAAVDMLYRGKYYNHSIDWQEGLGCKAAGFFSVFASELSVFTLTAITVERWHTITHALRLDRKLRLRHACIIMTAGWIFAAAAALLPTVGVSSYSKVSICLPMDVEDLVSQVYVVSLLLLNILAFLCVCGCYLSIYLTFRNPSSVPAHADTRVAQRMAVLIFTDFICMAPISFFAVSAALKTPLITVSESKVLLVLFYPINSCANPFLYAFFTRTFRRDFFFLAARFGLFKTRAQIYRTETSSCQQPAWTSSRSSRVTMYSLANTLSLDACVDSQSSKSW

SEQ ID NO: 19 - DNA, 2118 bp, Mugil cephalus
atgatgacggtgacgatactggtgatgctgaagtttgcgatgataaagatggaggcggcctcggcacctggctttgagctgggcttcagacccggacgggagaccgttttggccaaacaaacctgcttcctggtgggacccggggccatggcgataccctccaacatgtccagcagcacccagtgcttggaactcaagcagacacagatcagagtgattcccaagggtgccctcaccagcctgcagcacctcaggagactgaacatactgcagaatgacgaactgcagagtatcagtgatctcgccttcgtcggcctccctcgtctcacttacatcttaatatcagaaaatgttgctttagaaagaattggagcttccgccttctctgatctccctgaactcgttgagataaccataacaaagtcaagaaacctgcatttcatccactcagacgccttcagaaacctcatgaaactaaggtttatgaccatctcctacaccggcctgaaaatctttcccaacttttcccaggtccactccacctacaatgaatttcagcttcacctgcaggagaacagcgacttaaagaaagtccccgccaatgccttcagaggcctctgcactaaaactatcaagaagatatggctcaccagaaatggcatcaaggaggtggcaagtgacgctttcaatggaacaaagatgcaaagactatttctgaaaggcaatcggcagcttactcacatgagtcccaacgccttcgcgggttccagtgagttggaagtgcttgacatctcccaaacagcgctcagctctctgccaggctccatcctcgggggactcaagagactgatcgcagagtcagccaagaacctgaagaagcttcctcctcctcggttcttcaccaaactgcaccaggccaacctgacgtactcgtctcactgctgcgccttccagaacatgcagagaaacaaatcaaggaacaaccacaacctgtgcgctataccagaggcgaagaatgagcccttcttctacagggcgcactgctccacctccatcacctgcagcccggtgccggacgactttaacccgtgtgaggacatcatgtccgctgtccccttacgcatcctcatctggatcatctctatcctcgcgctactggggaacgcagtggttgtccttgtattgttgagcagcagatcgaagctcaccgttccccgtttcctcatgtgccacttggccttcgctgacctctgcatgggaatctacctggcagtcatagcagccgtagacatgttatatcgtggcaagtactacaaccactccatagactggcaggagggcttgggctgcaaggctgccggcttcttctcggtgtttgccagcgagctgtcggtgttcacgttgacggcgatcaccgtggagcgctggcacaccatcacgcatgccctgcgcctggaccgtaagcttcgcctgagacacgcctgcatcatcatgacggccggctggatcttcgccgcggcggccgcgcttctgcccacggtcggggtcagcagctacagcaaagtgagcatctgcttgcccatggatgtggaggatctggtgtcccaggtctatgtagtgtccctgctcctcctcaacatcctggccttcctctgtgtgtgcggctgctacctcagcatctacctgacattccgcaatccctcttcggtgccggcccacgccgacacgcgcatggctcaacgcatggccgtcctcatcgtcaccgacttcatctgcatggccccgatctccttcttcgccgtctcggccgcgctcaagacccccctcatcaccgtctcggaatctaaggtcctcctggtcctgttctaccccattaactcgtgcgccaaccccttcctgtacgccttcttcacccgcaccttccggcgggacttctttttcctggcggctcgcttcggcctgtttaagactcgggcgcagatttaccggacagagacctcttcctgtcagcagccagcatggacctcttcgaggagcagccgcgtgacaatgtactccttggccaacaccttgagcctggacgcgtgcgtagactcccagtcgtccaagtcatggtag

SEQ ID NO: 20 - protein, 705 aa, Mugil cephalus
MMTVTILVMLKFAMIKMEAASAPGFELGFRPGRETVLAKQTCFLVGPGAMAIPSNMSSSTQCLELKQTQIRVIPKGALTSLQHLRRLNILQNDELQSISDLAFVGLPRLTYILISENVALERIGASAFSDLPELVEITITKSRNLHFIHSDAFRNLMKLRFMTISYTGLKIFPNFSQVHSTYNEFQLHLQENSDLKKVPANAFRGLCTKTIKKIWLTRNGIKEVASDAFNGTKMQRLFLKGNRQLTHMSPNAFAGSSELEVLDISQTALSSLPGSILGGLKRLIAESAKNLKKLPPPRFFTKLHQANLTYSSHCCAFQNMQRNKSRNNHNLCAIPEAKNEPFFYRAHCSTSITCSPVPDDFNPCEDIMSAVPLRILIWIISILALLGNAVVVLVLLSSRSKLTVPRFLMCHLAFADLCMGIYLAVIAAVDMLYRGKYYNHSIDWQEGLGCKAAGFFSVFASELSVFTLTAITVERWHTITHALRLDRKLRLRHACIIMTAGWIFAAAAALLPTVGVSSYSKVSICLPMDVEDLVSQVYVVSLLLLNILAFLCVCGCYLSIYLTFRNPSSVPAHADTRMAQRMAVLIVTDFICMAPISFFAVSAALKTPLITVSESKVLLVLFYPINSCANPFLYAFFTRTFRRDFFFLAARFGLFKTRAQIYRTETSSCQQPAWTSSRSSRVTMYSLANTLSLDACVDSQSSKSW

SEQ ID NO: 21 - DNA, 10 bp, Mugil cephalus
  misc_feature (1)..(4): n is null
nnnnatgatg

SEQ ID NO: 22 - DNA, 16 bp, Mugil cephalus
gtgcttgtgagtacgc

SEQ ID NO: 23 - DNA, 15 bp, Mugil cephalus
catccacagggaact

SEQ ID NO: 24 - DNA, 16 bp, Mugil cephalus
gagactgtaagcttta

SEQ ID NO: 25 - DNA, 15 bp, Mugil cephalus
caactttaggaacat

SEQ ID NO: 26 - DNA, 16 bp, Mugil cephalus
ttacatgtgagtgtgt

SEQ ID NO: 27 - DNA, 15 bp, Mugil cephalus
ctgccccagcttaat

SEQ ID NO: 28 - DNA, 16 bp, Mugil cephalus
tgagatgtaagtataa

SEQ ID NO: 29 - DNA, 15 bp, Mugil cephalus
tcctttcagaaccat

SEQ ID NO: 30 - DNA, 16 bp, Mugil cephalus
gtttatgtgagtactc

SEQ ID NO: 31 - DNA, 17 bp, Mugil cephalus
tctcgtcacaggaccat

SEQ ID NO: 32 - DNA, 16 bp, Mugil cephalus
tcagctgtgagtctca

SEQ ID NO: 33 - DNA, 17 bp, Mugil cephalus
ttgacactcagtcacct

SEQ ID NO: 34 - DNA, 16 bp, Mugil cephalus
gaagatgtaagggctt

SEQ ID NO: 35 - DNA, 17 bp, Mugil cephalus
gctattgacagatggct

SEQ ID NO: 36 - DNA, 16 bp, Mugil cephalus
aagactgtgagtcaca

SEQ ID NO: 37 - DNA, 17 bp, Mugil cephalus
tttccccttagatttct

SEQ ID NO: 38 - DNA, 16 bp, Mugil cephalus
agtgctgtaagcatga

SEQ ID NO: 39 - DNA, 17 bp, Mugil cephalus
tgctcccacagtgacat

SEQ ID NO: 40 - DNA, 16 bp, Mugil cephalus
aaacaagtgcgcgtct

SEQ ID NO: 41 - DNA, 17 bp, Mugil cephalus
gtgtgtgctagatcaag

SEQ ID NO: 42 - DNA, 16 bp, Mugil cephalus
tgttgagtatgaaatg

SEQ ID NO: 43 - DNA, 17 bp, Mugil cephalus
tccattgtcaggcagca

SEQ ID NO: 44 - DNA, 16 bp, Mugil cephalus
ttctcggtgtgtacgc

SEQ ID NO: 45 - DNA, 17 bp, Mugil cephalus
gtcacgtgcaggtgttt

SEQ ID NO: 46 - DNA, 16 bp, Mugil cephalus
agcaaagtgggtgtta

SEQ ID NO: 47 - DNA, 17 bp, Mugil cephalus
tgttcgtgcaggtgagc

SEQ ID NO: 48 - DNA, 10 bp, Mugil cephalus
  misc_feature (10)..(10): n is null
cgtgcgtagn

SEQ ID NO: 49 - DNA, 37 bp, Mugil cephalus
gcgcgtggctcaacgcatggccgtcctcatcttcacc

SEQ ID NO: 50 - DNA, 240 bp, Mugil cephalus
tacctgacattccgcaatccctcttcggtgccggcccacgccgacacgcgcatggctcaacgcatggccgtcctcatcgtcaccgacttcatctgcatggccccgatctccttcttcgccgtctcggccgcgctcaagacccccctcatcaccgtctcggaatctaaggtcctcctggtcctgttctaccccattaactcgtgcgccaaccccttcctgtacgccttcttcacccgcacc

SEQ ID NO: 51 - DNA, 37 bp, Mugil cephalus
  misc_feature (5)..(5): a may be replaced by g
  misc_feature (32)..(32): t may be replaced by g
gcgcatggctcaacgcatggccgtcctcatcttcacc

SEQ ID NO: 52 - protein, 672 aa, Python bivittatus
MALFFLSVWLLLGACSGCQHPLCQCSDRMAFICQESEVTYVPRDIPRNSTELIFFLTKIRIIPKGAFAGFGEVEKIEISQNDALETIESDVFSHLPKLYEIRIEKASNLMYIDRHAFQNLPSLRYLLISNTAIRFLPAVNQVHTLQKVVLDIQDNINIRKIERNSFLGLSSDRVDIRLNKNGICEIENHAFNGTILSELNLSGNYNLEKLPNEVFQGAYGPGYLDISETKISQLPSIGLENINKLIAKSTYNLKKLPPLDKFHALIEANLTYPSHCCAFENWTKQNSMMHPICNKSSISEEPDFFELDENDYYHTLCREEFKVACFPEPDAFNPCEDIMGHIILRVLIWFINILAIMGNLVVFIILVSSQYKLTVPRFLMCNLAFADLCTGIYLLLIAIKDMQSRSQYYNYAIDWQTGAGCNTAGFFTVFASELSIYTLTVITLERWHTITYAMELDRKVRFRHAVVIMLVGWVFAFTVALLPIFEVSSYMKVSICLPMDIETLLAQAYIMFLLVLNILAFVIICTCYISIYFTVRNPNVFSSNSDTKIAKRMAILIFTDFLCMAPISFFAISASLKVPLITVSNSKILLVLFYPINSCANPFLYAIFTKTFRRDFFILLSKFGCCEMYAQIYRTETSSSVHTSHMKNGHYTPASKTSDGTIYSLVPLNHAN

SEQ ID NO: 53 - protein, 12 aa, Mugil cephalus
RVAQRMAVLIFT

SEQ ID NO: 54 - protein, 12 aa, Mugil cephalus
RMAQRMAVLIVT

SEQ ID NO: 55 - protein, 695 aa, Homo sapiens
MALLLVSLLAFLSLGSGCHHRICHCSNRVFLCQESKVTEIPSDLPRNAIELRFVLTKLRVIQKGAFSGFGDLEKIEISQNDVLEVIEADVFSNLPKLHEIRIEKANNLLYINPEAFQNLPNLQYLLISNTGIKHLPDVHKIHSLQKVLLDIQDNINIHTIERNSFVGLSFESVILWLNKNGIQEIHNCAFNGTQLDELNLSDNNNLEELPNDVFHGASGPVILDISRTRIHSLPSYGLENLKKLRARSTYNLKKLPTLEKLVALMEASLTYPSHCCAFANWRRQISELHPICNKSILRQEVDYMTQARGQRSSLAEDNESSYSRGFDMTYTEFDYDLCNEVVDVTCSPKPDAFNPCEDIMGYNILRVLIWFISILAITGNIIVLVILTTSQYKLTVPRFLMCNLAFADLCIGIYLLLIASVDIHTKSQYHNYAIDWQTGAGCDAAGFFTVFASELSVYTLTAITLERWHTITHAMQLDCKVQLRHAASVMVMGWIFAFAAALFPIFGISSYMKVSICLPMDIDSPLSQLYVMSLLVLNVLAFVVICGCYIHIYLTVRNPNIVSSSSDTRIAKRMAMLIFTDFLCMAPISFFAISASLKVPLITVSKAKILLVLFHPINSCANPFLYAIFTKNFRRDFFILLSKCGCYEMQAQIYRTETSSTVHNTHPRNGHCSSAPRVTSGSTYILVPLSHLAQN

SEQ ID NO: 56 - protein, 692 aa, Mus musculus
MALLLVSLLAFLGSGSGCHHWLCHCSNRVFLCQDSKVTEIPPDLPRNAIELRFVLTKLRVIPKGSFSGFGDLEKIEISQNDVLEVIEADVFSNLPNLHEIRIEKANNLLYINPEAFQNLPSLRYLLISNTGIKHLPAFHKIQSLQKVLLDIQDNINIHIIARNSFMGLSFESVILWLNKNGIQEIHNCAFNGTQLDELNLSDNNNLEELPDDVFQGASGPVVLDISRTKVYSLPNHGLENLKKLRARSTYRLKKLPSLDKFVMLIEASLTYPSHCCAFANWRRQTSELHPICNKSISRQDIDDMTQPGDQRVSLVDDEPSYGKGSDMLYSEFDYDLCNEFVDVTCSPKPDAFNPCEDIMGYNILRVLIWFISILAITGNTTVLVVLTTSQYKLTVPRFLMCNLAFADLCIGIYLLLIASVDIHTKSQYHNYAIDWQTGAGCDAAGFFTVFASELSVYTLAAITLERWHTITHAMQLECKVQLCHAASIMVLGWAFAFAAALFPIFGISSYMKVSICLPMDIDSPLSQLYVMALLVLNALAFVVICGCYTHIYLTVRNPNIVSSSRDTKIAKRMATLIFTDFLCMAPILFFAISASLKVPLITVSKAKILLVLFYPINSCANPFLYAIFTKNFRRDFFVLMSKFGCYEVQAQIYKTETSSITHNFHSRKNPCSSAPRVTNSYVLVPLNHSVQN

SEQ ID NO: 57 - protein, 701 aa, Oreochromis niloticus
MMLVMTLMMLLIVTIKMAAASAHGSEMDIRPGFHPSLAKQTSCLSYQVMFGVTAFPSNISNAQCLEVKQTQIREIQQGTLSSLQHLMELTISENDLLESIGAFAFSGLPHLTKILISKNAALRNIGAFVFSNLPELSEIIITKSKHLSFIHPDAFRNMARLRFLTISNTGLRIFPDFSKIHSTACFLLDLQDNSHIKRVPANAFRGLCTQTFAEIRLTRNGIKEVASDAFNGTKMHRLFLGGNRQLTHISPNAFVGSSELVVLDVSETALTSLPDSILDGLKRLIAESAFNLKELPPIQLFTKLHQAKLTYPSHCCAFLNMHRNRSRWHSLCDNPEAKNNLHFFREYCSNSTNITCSPAPDDFNPCEDIMSATPLRILIWIISVLALLGNAVVLLVLLGSRYKLTVPRFLMCHLAFADLCMGIYLVVIATVDMLTRGRYYNYAIDWQMGLGCNAAGFFTVFASELSVFTLTAITVERWHTITHALRLDRKLRLRHACIIMTIGWIFSLLAALLPTVGISSYGKVSICLPMDVESLVSQFYVVCLLLLNILAFFCVCGCYLSIYLTFRKPSSAAAHADTRVAQRMAVLIFTDFICMAPISFFAISAALKLPLITVSDSKLLLVLFYPINSCSNPFLYAFFTRNFRRDFFLLAARFGLFKTRAQIYRTESSSCQQPTWTSPKNSRVILYSLVNTLSLDGKQEC

SEQ ID NO: 58 - protein, 688 aa, Seriola dumerili
MMSVIMLMIMVMNTVTASAPGSEMHIKPGGCHKLGSGVTEIPSNIPSNTQCLRVKQTQIREIPQGALTSLQHLRKLIIMKNDVLESISASAFANLSQLTYIIISENLALERIEAFAFSNLPELTEINIEKSKHLSSIHPDAFGNITKLQHLTISHTTLRIFPNFSKIQSTATELLFQLQENSHIERVPANAFRGLCTQAILEIRLTRNGIKEVASDAFNGTRMKKLSLRGNKHLTHINPNAFVGSSELVVLDVSHTTVTSLPDSILGGLQRLFAESALELKKLPSLQLTKLHQASLTYQSHCCAFQNIRRNRSRWNHLCSNPNAPDQLHFYREHCSNFTSITCSPAPDAFNPCEDIMSAVPLRVLIWIISILALLGNAVVLLVLIGSRSKLTVPRFLMCHLAFADLCMGIYLAVIATVDMFTRGRYYNDAIDWQMGLGCSAAGFFTVFASELSVFTLTAITLERWHTITHALRLDRKLRLRHASIVMTAGWIFSSLAALLPTVGVSSYSKVSICLPMDVEFLASQVYIVSLLLLNILAFFCVCGCYLSIYLTVRNPSSAPAHADTRVAKRMAILIFTDFICMAPISFFAISAALKLPLITVSQSKFLLVFFYPINSCSNPFLYAFCTRTFRRDFFFLAARFGLFKTRAQIYRTESSSCQQPAWTSPKSSHVVLYSLANALSLDVKQEC

SEQ ID NO: 59 - protein, 702 aa, Dicentrarchus labrax
MMMVMILIMLMILMIKTATASVPGPEMDVKPGVETSLAKRTLSFCYQLKFGVTEIPSSISSNTTCLEVKQTEIVVIPQGALNSLQHLRKLTIWENDKLESINEFAFASLSQLTDIFISGNVALKNIGAFAFSDLPELTEITITKSKHLTHINPDAFKDIVKLKYLTIANTGLRLFPDFTKIHSTGLLLFDLHDNSHIERVPANAFKGLCTQTIPEIRLTRNGIKEVASDAFNGTKMHRLFLRGNKQLTHINPNAFVGSSELVVLDISQTALSSLPDYILGGLQKLIAESAPNLKELPPLELFTKLHQANLTYSSHCCAFHNIHRNRSKWNSLCSHPDAQGNLHFYRDYCSNSTSIICTPTQDDFNPCEDIMSAVPLRVLIWIISILALLGNTVVLLVLLGSRTKLTVPRFLMCHLAFSDLCMGIYLVVIATVDMLTQGQYYNHAIDWQTGLGCSVAGFFTVFASELSVFTLTAITLERWHTITHALRLDRKLRLRHACIVMTAGWIFSAVAALLPTVGVSSYGKVSICLPMDVEFLGSQVYVVSLLLLNILAFFCVCGCYLSIYLTVRNPSSAPAHADTRVAQRMAVLIFTDFVCMAPISFFAISAALKLPLITVSDAKLLLVLFYPINSCSNPFLYAFFTRTFRRDFFLLAARFGLFKTRAQIYRTESSSCQQPAWTSPKSSHVMLYSLANALSLEGKPEF

SEQ ID NO: 60 - protein, 659 aa, Oncorhynchus mykiss
MMKMKKIMKMLLCMLGCVCVSQAEVAMVNSGTTFTYLCMGNTITHMPTHIPKNTTDLEFKQTHIRVFPREAFTNLQQLTAIVLTENGMLESIGAFAFANLPRLTEITITKSKHLVIIHHQAFIGLPKLSHLTICNTGLRVLPNFSRIHSAALTFLLDLQDNVHIVIIPSNAFLGLTTNTIDELRLTKNGISEVESHAFNGTKIHKLFLMGNLQLSHMHNNSFKGAEGPGFLDISRTALSSLPESVLGEVEHLSAVSVFSLRALPPLSLFTKLRQANLTYPSHCCAFHKHQRNRTFRMTSACFKPGAQNNLHFFMDFCLNWTSVACSPAPDAFNPCEDIMGSAPLRVLIWITSVLALLGNTIVLLVLLGSRAKMTVPRFLMCHLSFADLCMGVYLVVIATVDVRTRGLYYNHAISWQTGAGCDIAGFFTVFASELSVFTLTAITLERCHTITHALRLDRKLRLRHACAVMATGWAFSCLAALLPTVGVSSYSKVSICLPMDVESLPSQVFVMFLLLLNVVAFLCVCVCYLSIYLSVRNSSSPPASAETSRMAQRMAILIFTDFLCMAPISFFALSAALKLPLITVSDSKLLLVLLYPINSCANPFLYGLCTRTFRRDFFLLAARYGLFTTKAQVYRTESFPVQQAAWIQMSPKVSHGTLC

SEQ ID NO: 61 - protein, 694 aa, Takifugu rubripes
MAPRAVWLLVALSAVLNVRSCCAFSCPAICRCSQHTFQCSRDTQLASRPAARAVPRLRLTHMPLKEVPTHAFKELINITAIEISQSDSIAQIQRHAFLSLHSLAQISVRNINSLRVIEKGAFTDLPRLEHLSICNTGLIHFPDFTTVSSLAANIILEIADNMRIESIPANSFQGIAEESVDIRNLVRNGFRTIRSHAFNGTKLNTLILKDNRHLQDIQEDAFEGAAGPTVLDVSSTALSSLPPRGLRQVTTLKATLTFALKTLPPLESLADLLEAELTYPSHCCAFHAWRRKQRESSLKNLTKLCNLNANEIESAAEDAALSDYISFQYPDLDLYCLNNPLVKCTPKPDAFNPCEDLLGFSFLRSLTWIITACAVTGNLVVLVILLASHSKITISRFLMCNLAAADLCMGLYLMLIAFMDFQSRHQYYNHATDWQTGPGCGTAGFLTVFASELSVYTLTVISLERWHTIANAMRVNKRLRLRHVALIMAAGWAFSLLVALLPLVGVSSYSKVSICLPMDINGPGSQLYVVAVLVLNVVAFLVVCYCYACIYLSVRNPEHATRHGDTKMAKRMAVLIFTDFVCMAPISFFAISAALRMPLITVSHSKILLILFYPINSLCNPFLYTLFTRAFRKDLFRLLRRCSCWRSSADFCRSMKPPPPPPAESGQKKAGRKSLSFYAYHIKMNDCFLNKGTT

SEQ ID NO: 62 - protein, 59 aa, Homo sapiens
IHIYLTVRNPNIVSSSSDTRIAKRMAMLIFTDFLCMAPISFFAISASLKVPLITVSKAK

SEQ ID NO: 63 - protein, 59 aa, Mus musculus
THIYLTVRNPNIVSSSRDTKIAKRMATLIFTDFLCMAPILFFAISASLKVPLITVSKAK

SEQ ID NO: 64 - protein, 59 aa, Mugil cephalus
LSIYLTFRNPSSVPAHADTRVAQRMAVLIFTDFICMAPISFFAVSAALKTPLITVSESK

SEQ ID NO: 65 - protein, 59 aa, Mugil cephalus
LSIYLTFRNPSSVPAHADTRMAQRMAVLIVTDFICMAPISFFAVSAALKTPLITVSESK

SEQ ID NO: 66 - protein, 59 aa, Seriola dumerili
LSIYLTVRNPSSAPAHADTRVAKRMAILIFTDFICMAPISFFAISAALKLPLITVSQSK

SEQ ID NO: 67 - protein, 59 aa, Dicentrarchus labrax
LSIYLTVRNPSSAPAHADTRVAQRMAVLIFTDFVCMAPISFFAISAALKLPLITVSDAK

SEQ ID NO: 68 - protein, 59 aa, Oreochromis niloticus
LSIYLTFRKPSSAAAHADTRVAQRMAVLIFTDFICMAPISFFAISAALKLPLITVSDSK

SEQ ID NO: 69 - protein, 60 aa, Oncorhynchus mykiss
LSIYLSVRNSSSPPASAETSRMAQRMAILIFTDFLCMAPISFFALSAALKLPLITVSDSK

SEQ ID NO: 70 - protein, 59 aa, Takifugu rubripes
ACIYLSVRNPEHATRHGDTKMAKRMAVLIFTDFVCMAPISFFAISAALRMPLITVSHSK

SEQ ID NO: 71 - protein, 692 aa, Xenopus tropicalis
MPLIFVCMLFCLQPCFGCHAVCQCMGRSFVCQENKMEQIPNNIPRNATELRFMLTKITIIPNGAFAGFQDLEKIEISQNDVLTSIEANVFSNSPQLHEIRIEKANSLVYIDQEAFQNLPKLKYLLISNTGIHFIPVVSKIQSLQMVLLEIQDNINIKKIERNSFAGLSIDNVILRLNKNGIEEIQNYAFNGTNLNELILSGNERLARLPDLVFHGSTGPTVLDISNTRISSLPSFGLEKIKRLKAKSAYNLKKLPALEKFEELIEADLTYPSHCCAFANWKREITDWDPICNKTYESQPEDLKSLNKRSAQVDPILSNGETTYDLIDKEDDIAFDYFFCYGKRDFVCSPTPDEFNPCEDIMGYTALRVLIWCVNILAITGNSVVLIILLTSQYKFTVPRFLMCNLAFADLCMGVYLLLIAAVDIKTKSQYYNYAIDWQTGAGCHAAGFFTVFASELSVFTLTVITLERWHTITYAMQLDRKVRFRHATMIMASGWVFSFTVALLPIVGVSSYMKVSICLPMDIENPLSQAYIMFLLVLNVLAFVVICTCYIGIYLTVRNPDVISSNSDTKIAKRMAILIFTDFLCMAPISFFAISASLKIPLITVSKSKILLVLFYPINSFANPFLYAIFTKTFRRDFYILMSKFGCCEARAQIFRTETSSSAHNLHARNGHFVPGPKSSSGSVYTLVPLNH

SEQ ID NO: 72 - protein, 704 aa, Anas platyrhynchos
MEDKGADVKTEMFLVLTCSLILLASCSSCQHHTCHCAGRIFICQESKVVQLPRDIPTNATELRFVLTKMRVIPKGAFAGLHDLEKIEISQNDALEVIEANVFSNLPKLHEIRIEKANNLVYIDQDAFQHLPSLRYLLISNTGLRFLPVVQKVHSFQKVLLDIQDNINIRTIERNSFMGLSSESVILWLNKNGIQEIENHAFNGTYLDELNLSDNQNLEKLPNDVFQGANGPVVLDISRTKISFLPGHGLELIKKLRARSTYNLKKLPDLSKFRSLIEANFTYPSHCCAFTNWKRQNTELHPICSISQAKQDLDEQPGNKIHRRSAAEDYISNYAIGFDPSENEFDYGLCNEVVNVACSPKPDAFNPCEDIMGYNILRVLIWFISILAITGNIVVLIILISSQYKLTVPRFLMCNLAFADLCIGIYLLFIASVDIQTKSQYYNYAIDWQTGAGCNAAGFFTVFASELSVYTLTVITLERWHTITYAMQLDRKVRFRHAVIIMIFGWMFAFTVALLPIFGVSSYMKVSICLPMDIETPFSQAYVIFLLVLNVLAFVIICACYICIYFTVRNPNVISSNSDTKIAKRMAILIFTDFLCMAPISFFAISASLKVPLITVSKSKILLVLFYPINSCANPFLYAIFTKTFRRDFFILLSKFGCCEMQAQIYRTDTSSSAHNFHTRNGHYPPASKNSDGTIYSLVPLNHLN

(19) United States
(12) Patent Application Publication
(10) Pub. No.: US 2023/0242984 A1
(43) Pub. Date: Aug. 3, 2023

(54) SEX DETERMINATION MARKERS IN MULLETS AND METHODS OF USING THEREOF

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Michael RON, Nes Ziona (IL); Andrey SHIRAK, Petach Tiqua (IL); Eyal SEROUSSI, Sharey-Tikva (IL); Hanna ROSENFELD, Eilat (IL); Iris MEIRI ASHKENAZI, Eilat (IL)

(21) Appl. No.: 18/004,788

(22) PCT Filed: Jul. 7, 2021
(86) PCT No.: PCT/IL2021/050834

§ 371 Date: Jan. 9, 2023

(30) Foreign Application Priority Data
Jul. 8, 2020 (IL) 275926

(51) Int. Cl. C12Q 1/6879; A01K 67/02; A61K 31/568; A01K 61/10

(52) U.S. Cl. CPC C12Q 1/6879; A01K 67/02; A61K 31/568; A01K 61/10; A01K 2227/40; A01K 2207/20; C12Q 2600/156

(57) ABSTRACT

The invention relates to a sex determination (SD) marker of a male Mugilidae fish comprising a nucleic acid sequence corresponding to a sequence of a Follicle-stimulating hormone receptor (FSHR) gene of said fish and including at least one male (Y) specific nucleotide difference when compared to a corresponding sequence on a FSHR gene of a female Mugilidae fish. The invention further concerns uses of this sex determination marker in methods of early sex determination in a Mugilidae fish, methods of sex-reversal of a Mugilidae fish prior to appearance of a sex phenotype, and methods for producing a mono-sex population of Mugilidae fishes.

### TECHNOLOGICAL FIELD

The invention relates to methods for early sex determination in fish, specifically the flathead grey mullet and methods for producing mono-sex populations of a preferred gender of such fish.

### BACKGROUND ART

References considered to be relevant as background to the presently disclosed subject matter are listed below:
[1] Pan B S, Sheen S-S, Shew S-H, Chen C-M, Hung J (1992). Difference in Sex Ratio and Physiological Indices of Cultured and Wild Grey Mullet Mugil cephalus in Taiwan. Nippon SUISAN GAKKAISHI 58: 1229-1235.
[2] Dor L, Shirak A, Rosenfeld H, Ashkenazi I M, Band M R, Korol A, et al. (2016). Identification of the sex-determining region in flathead grey mullet (Mugil cephalus) Anim Genet 47: 698-707.
[3] Murozumi N, Nakashima R, Hirai T, Kamei Y, Ishikawa-Fujiwara T, Todo T, et al. (2014). Loss of Follicle-Stimulating Hormone Receptor Function Causes Masculinization and Suppression of Ovarian Development in Genetically Female Medaka. Endocrinology 155: 3136-3145.

Acknowledgement of the above references herein is not to be inferred as meaning that these are in any way relevant to the patentability of the presently disclosed subject matter.

### BACKGROUND

Sex determination (SD) is variable and can involve genetic, environmental, and social factors. In most cases a single gene plays a role of a master key regulator (MKR) initiating the dichotomic division of sexes (males and females). SD among different fishes is extremely variable, even between families of the same genus, for which different MKRs have been found.

The flathead grey mullet (Mugil cephalus) is the most widely spread fish among Mugilidae family. Despite its abundancy and the fact that it is adaptive to a wide range of environments, it is not fully domesticated and fry production for aquaculture is mainly dependent on collection from river estuaries. The high price of female products and their faster growth rate increase the value of all-female population production.

Utilizing sex steroids in larvae feed can cause sex inversion in many fish species and is often used in commercial production. Technologies for mono-sex production through parent sex-reversal can reduce environmental and food safety concerns caused by mono-sex production via direct larvae feeding. Researchers striving to increase the female ration of cultured mullets have shown that sex reversal can be manipulated in males up to age of one year by oral administration of the 17β-estradiol [1]. Dor et al (2016) analyzed the sex of M. cephalus progeny in two independent families and showed (based on genetic markers) that the alleles linked with maleness were transmitted from the fathers, thus indicating that mullets have a genomic XX/XY SD system [2].

The SD region of M. cephalus was mapped to a 1.8 Mbp region, based on synteny with Nile tilapia (Oreochromis niloticus) genome [2]. One of the genes comprised in this region and showing sex biased gene expression is Follicle-stimulating hormone receptor (FSHR), and thus is a candidate MKR gene for SD based on its putative position and relevant function in sex differentiation [3].

### SUMMARY OF THE INVENTION

The present disclosure provides, in accordance with a first of its aspects, a sex determination (SD) marker of a male Mugilidae fish comprising a nucleic acid sequence corresponding to a sequence of a Follicle-stimulating hormone receptor (FSHR) gene of said fish and including at least one male (Y) specific nucleotide difference when compared to a corresponding sequence on a FSHR gene of a female Mugilidae fish.

The present disclosure provides, in accordance with a second of its aspects, a method of sex determination in a Mugilidae fish before appearance of a sex phenotype, the method comprises determining presence of a SD marker of a male Mugilidae fish, the SD marker comprising a nucleic acid sequence that corresponds to a sequence of a FSHR gene of the Mugilidae fish and includes at least one male specific nucleotide difference when compared to a corresponding sequence on a FSHR gene of a female Mugilidae fish;

wherein presence of said SD marker being indicative that the fish is a male Mugilidae fish.

In yet a third of its aspects, the present disclosure provides a method of sex-reversal of a Mugilidae fish prior to appearance of a sex phenotype, the method comprising:
(a) identifying in the fish presence or absence of a sex determination (SD) marker of the male Mugilidae fish, the SD marker comprising a nucleic acid sequence that corresponds to a sequence of a FSHR gene of the Mugilidae fish and includes at least one male specific nucleotide difference (Y) when compared to a corresponding sequence on a FSHR gene of a female Mugilidae fish (X); and
(b) treating fish with a sex reversal steroid selected according to presence or absence of said nucleic acid sequence,

wherein when said SD marker is present, the fish is treated with a sex reversal steroid causing sex reversal of the treated XY fish from a male phenotype into a female phenotype; and

wherein said SD marker is absent, the fish is treated with a sex reversal steroid causing sex reversal of the XX treated fish from a female phenotype into a male phenotype.

In accordance with a fourth of its aspects, the present disclosure provides a method for producing a mono-sex population of female Mugilidae fishes, the method comprising:
(a) providing a Mugilidae fish at an age prior to appearance of a sex phenotype;
(b) identifying in said fish presence or absence of a sex determination (SD) marker of a male Mugilidae fish, the SD marker comprising a nucleic acid sequence that corresponds to a sequence of a FSHR gene of the Mugilidae fish and includes at least one male specific nucleotide difference when compared to a corresponding sequence on a FSHR gene of a female Mugilidae fish;
(c) selecting fish that is absent of said SD marker;
(d) treated the selected fish with an amount of a male sex steroid, the amount being effective to cause sex reversal of the fish into a reversed male fish (can be defined as a male XX); and
(c) allowing said reversed male fish (XX) to mate with a female fish (XX, preferably native female fish), to thereby obtain a mono-sex population of female fishes.

In accordance with yet a fifth of its aspects, the present disclosure provides a mono-sex population of Mugilidae fishes, preferably female mono-sex population, wherein said fishes are at an age before appearance of a sex phenotype, as well as a fish cultivating reservoir comprising the mono-sex population of fish.

In accordance with yet a sixth of its aspects, the present disclosure provides a method of sex-reversal of a Mugilidae fish, comprising introducing in a fish embryo a gene-editing agent for modifying a sex determination marker of said fish, wherein said sex determination marker comprises a nucleic acid sequence corresponding to a sequence of an FSHR gene of said fish and including at least one male (Y) specific nucleotide difference when compared to a corresponding sequence on an FSHR gene of a female (XX) Mugilidae fish.

Finally, the present disclosure provides a nucleic acid sequence for use as a sex determination (SD) marker of a male Mugilidae fish, the nucleic acid sequence corresponding to a sequence of a Follicle-stimulating hormone receptor (FSHR) gene of said fish and including at least one male specific nucleotide difference when compared to a corresponding sequence on a FSHR gene of a female Mugilidae fish.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1E provide the Sanger sequencing of the sex associated region of FSHR in exon 14, after amplification using PCR primers (pair 1, Table 1). Single Nucleotide Polymorphism (SNPs) (nucleotide positions 1732 and 1759 of the predicted coding sequence (cds)) are marked by an arrow. FIG. 1A provides the sequence relating to the Female Reverse and is also denoted by SEQ ID NO: 49. FIG. 1B provides the sequence relating to the Female Forward and is also denoted by SEQ ID NO: 49. FIG. 1C provides the sequence relating to the Male Reverse and is also denoted by SEQ ID NO: 51; and FIG. 1D provides the sequence relating to the Male Forward and is denoted by SEQ ID NO: 51. FIG. 1E provides the FSHRX (upper) and FSHRY (lower) putative translated amino acids and are denoted by SEQ ID NO: 53 and 54, respectively. Partially conserved and highly conserved amino acid residues are indicated by a grey and black background, respectively. The white box indicates the novel male (Y)-specific amino acid substitution between the proteins (following the coloration presented in FIG. 2).

FIG. 2 provides a comparison between predicted FSHR of M. cephalus and other vertebrate FSHRs (GenBank: Human NP_000136; Mouse NP_038551; Tilapia NP_001266517; Seriola XP_022599280; Labrax AAV48628; Trout NP_001117799; Fugu XP_029691459 as denoted by SEQ ID NO: 55, 56, 57, 58, 59, 60, 61 respectively) Amino acid numbering follows the complete gene alignment. The highly conserved phenylalanine (F) residue (nt 1759 in exon 14, Table 3) that was uniquely altered in FSHR of M. cephalus male is marked with an arrow indicating the corresponding human amino acid position (579, Human) The protein domains are indicated above the amino acid alignment. Identical and similar amino acid residues in at least two of four sequences are indicated by a black and grey background, respectively. White boxes indicate non-conservative amino acid changes between the proteins. The amino acid sequences relating to Human, Mouse, Mugil Female, Mugil Male, Seriola, Lavrax, Tilapia, Trout and Fugu are as denoted by SEQ ID NO: 62, 63, 64, 65, 66, 67, 68, 69 and 70 respectively.

FIG. 3 provides a graph showing expression data for FSHRX in terms of normalized Trimmed Mean of values (TMM) for 28 days post hatch larvae groups: A (“XX”) and B (“XY”), for adult brains and for gonads of both sexes. Standard error bar is shown at the top of columns with replicates.

### DETAILED DESCRIPTION OF THE INVENTION

Deciphering the SD mechanism of M. cephalus has great significance for mullet aquaculture as breeding of all-female population is expected to improve, inter alia, production.

Specifically, as disclosed herein, a high association was found to exist between a haplotype of non-synonymous SNPs in Follicle-Stimulating Hormone Receptor (FSHR) gene, and specifically, although not exclusively, in at least one of the two SNP, c.1732G>A and c.1759T>G, and sex in M. cephalus. It was thus concluded that such SNPs in FSHR were thus considered to act as SD markers of male Mugilidae fish.

Based on the current finding, the present disclosure provides, in accordance with a first of its aspects, a sex determination (SD) marker of a male Mugilidae fish comprising a nucleic acid sequence corresponding to a sequence of a Follicle-stimulating hormone receptor (FSHR) gene of said fish and including at least one male (Y) specific nucleotide difference when compared to a corresponding sequence on a FSHR gene of a female Mugilidae fish.

The Mugilidae fish are a family of ray-finned fishes also named mullets or grey mullets, found worldwide in coastal temperate and tropical waters, and some species in fresh water. The family includes about 78 species in 20 genera. Mullets are distinguished by the presence of two separate dorsal fins, small triangular mouths, and the absence of a lateral line organ. A particular member of this family, according to the present disclosure belongs to the genera Mugil, as further discussed below.

In the following disclosure, a “sex determination (SD) marker” relates to a region in the genome of an organism which enables to determine the development of sexual characteristics in the organism, thereby defining the organism gender.

There are two main sex-determination systems in eukaryotic species which are the XY sex-determination system and the ZW sex-determination system (e.g., XY or ZW). In the XY sex-determination system, the male is a heterogametic organism and the gender-chromosome specific for the heterogametic gender is the Y chromosome while the gender-chromosome specific for the homogametic gender is the X chromosome. In the present disclosure, the inventors have determined sex-biased polymorphisms, which allow assembling the male specific and nonspecific sequences of the FSHR, representing the “Y” and “X” genomic variants, designated as FSHRY and FSHRX.

As used herein, a “at least one male specific nucleotide difference” relates to at least one specific nucleotide in the FSHR gene which is found only in the male, i.e. is different from the corresponding nucleotide in the female sequence, and is thus concluded to be associated with sex determination.

As used herein, when referring to “sequence corresponding to a sequence” it is to be understood that the comparison between the sequence, so as to determine a difference in one or more nucleotides, is conducted when the two sequences are optimally aligned.

The Examples of the present disclosure show that in order to determine if a nucleotide difference is specific to a male phenotype, the sequence of both male and female FSHR genes is compared by analyzing paired-reads sequences, and the presence of one or more nucleotide differences between the two genders is determined.

In some examples, the at least one nucleotide difference is one encoding for at least one amino acid change between the female and male phenotype of Mugilidae fish and is determined to be associated with SD of the Mugilidae fish.

Differences between sequences, and in other words, SD markers can be determined by techniques known in the art. In some cases, Polymerase Chain Reaction (PCR) is used to determine sequence differences.

The male specific nucleotide difference disclosed herein also corresponds to the XY/XX SD model described above and in other words, in accordance with the present disclosure, it is present only in one copy (Y) in male (XY) and not present in female at all (XX) Amino acid alignment with homologous regions of other vertebrate species may enable also to determine non-conservative amino acid substitutions.

In accordance with the present disclosure, the at least one nucleotide difference being associated with the SD marker is present in the FSHR gene. The FSHR is a transmembrane receptor that interacts with the follicle-stimulating hormone (FSH) and represents a G protein-coupled receptor (GPCR). Without being bound by theory, FSH/FSHR role in sex hormones biosynthesis may possibly influence SD in Mugilidae fish and specifically M. cephalus.

In some examples, the Mugilidae fish is preferably Mugil cephalus, or in short, M. cephalus.

In some examples, when the male fish is M. cephalus the FSHR gene comprises a nucleic acid sequence denoted by SEQ ID NO: 11.

In some examples, the FSHR gene of the male M. cephalus comprises a nucleic acid sequence that has high homology, e.g. at least 80%, at times, at least 90%, or even at least 95% or 99% homology with SEQ ID NO: 11, when optimally aligned therewith, and comprises the at least one male specific nucleotide difference as disclosed herein.

In some examples, when the male fish is M. cephalus, the FSHR gene is one encoding for an amino acid sequence comprising SEQ ID NO: 20.

In some examples, when the male fish is M. cephalus, the FSHR gene encodes for an amino acid sequence that has high homology, e.g. at least 80%, at times, at least 90%, or even at least 95% or 99% homology with SEQ ID NO: 20, when optimally aligned therewith, and includes at least one amino acid difference that is associated with SD of the fish.

As used herein, “high homology” or “X % homology” denotes the percent of identity of a homologous sequence with the reference sequence (e.g. the native sequence of the organism as determined by PCR, such as SEQ ID NO:20) when both are optimally aligned or % identity of a sequence within a comparison window of the reference sequence, with the condition that the homologous sequence includes at least the SD marker (i.e. it includes the replacement(s) identified as being associated with sex determination). A high homology should be understood as one in which there is at least 80% identity with the reference sequence, at times, at least 85% identity, or 90% identity, or 95% identity or even 97%, 98% and 99% identity with the reference sequence.

In the context of the present disclosure, the comparison window denotes a conceptual segment of the FSHR sequence comprising at least 20 contiguous nucleotides and including the at least one nucleotide difference when compared to the corresponding female sequence; at times, between 20 and 50 contiguous nucleotides, at times between 20 and 100 contiguous nucleotides or even between 20 to 150 contiguous nucleotides. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol., 48:443, by the search for similarity method of Pearson and Lipman (1988) “Proc. Natl. Acad. Sci. (U.S.A.) 85: 2444, and by computerized implementations of these algorithms.

In some examples, the comparison region is a region of less than 150, 100, 95, 90, 85, 80, 75, 70, 65, 60, 55, 50, 45, 40, 35, 30, 25, 20, 15, 10 or 5 contiguous base pairs (bp). In some specific examples, the two or more male specific nucleotide differences are within a comparison window/region of 20-35, or 25-30 contiguous nucleotides.

In some examples, the female fish is M. cephalus and the female fish comprises a nucleic acid sequence denoted by SEQ ID NO: 13.

In some examples, the female fish is M. cephalus and the female fish FSHR gene encodes for an amino acid sequence comprising SEQ ID NO: 14.

Without being limited thereto, the Examples provided herein and which form an independent part of the present disclosure, show that the SD associated haplotype on FSHR gene is found in regions encoding for two adjacent protein domains transmembrane α-helices six (TM6), and intracellular loop 3 (IL3) which are both part of the G protein interaction domain loop of the FSHR protein. Thus, in accordance with some examples, the SD marker of the present disclosure corresponds to a sequence encoding a G protein interaction domain loop of the FSHR protein. In some specific embodiments, the SD marker corresponds to a sequence encoding at least one of transmembrane α-helices six (TM6) domain and intracellular loop 3 (IL3) domain of FSHR protein.

In some examples, the at least one male (Y) specific nucleotide difference of the present disclosure is a Single-Nucleotide Polymorphism (SNP).

As used herein, a “Single-Nucleotide Polymorphism (SNP)” refers to a substitution of a single nucleotide at a specific position in the genome, that is present in a sufficiently large fraction of the population e.g. 1% or more, specifically about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85%, about 85% to 90%, about 90% to 95%, about 95% to 99%, or about 99% to 99.9%, 100% or more.

In some examples, the at least one male (Y) specific nucleotide difference or SNP of the present disclosure falls within a particular codon of the coding sequence of the FSHR gene. As appreciated, a codon is a series of three nucleotides (a triplet) that encodes a specific amino acid residue in a polypeptide chain or for the termination of translation (stop codons). There are 64 different codons (61 codons encoding for amino acids and 3 stop codons) but only 20 different translated amino acids. The overabundance in the number of codons allows many amino acids to be encoded by more than one codon. In the context of the present disclosure, the first and second nucleotides of a codon triplet are more significant for amino acid translation, than the third nucleotide within the triplet.

In some examples, the at least one nucleotide difference is located at a first nucleotide position within a codon.

In some other examples, the at least one nucleotide difference is located at a second nucleotide position within a codon.

In some examples, the nucleic acid disclosed herein comprises at least two nucleotide differences with the FSHR gene, when the male FSHR gene is compared with the corresponding sequence of the female FSHR gene.

In some examples, the at least one nucleotide difference comprises a haplotype of two non-synonymous SNPs in FSHR gene.

Thus, in some examples, the SD marker or the nucleic acid sequence for use of the present disclosure comprises two or more Y specific nucleotide differences, when compared to the corresponding sequence of the FSHR gene of a female Mugilidae fish.

In some examples, the two or more male (Y) specific nucleotide differences are within comparison window/a region of less than 150 contiguous base pairs (bp).

In some examples, the two or more male specific nucleotide differences are within a comparison window/region of less than 100, 95, 90, 85, 80, 75, 70, 65, 60, 55, 50, 45, 40, 35, 30, 25, 20, 15, 10 or 5 contiguous base pairs (bp). In some specific examples, the two or more male (Y) specific nucleotide differences are within a comparison window/region of 20-35, or 25-30 contiguous nucleotides.

In some examples, the SD marker or the nucleic acid sequence for use according to the present disclosure corresponds to a sequence from Exon 14 of the FSHR gene.

As shown in the non-limiting Examples, by investigating polymorphism in mullet's FSHR using DNA sequencing and comparing its gene sequence between males and females, the inventors have identified two specific SNPs capable of encoding non-conservative amino acid substitutions, c.1732G>A replacing a valine with methionine and c.1759T>G replacing a phenylalanine with valine.

Thus, in accordance with some examples, the SD marker or the nucleic acid sequence for use according to the present disclosure comprises within a comparison window/region, such as a region of less than 150 bp, at least one of (i) a Thymine (T) nucleotide instead of a Guanine (G) nucleotide present in the female corresponding sequence (ii) a Guanine (G) nucleotide instead of an Adenosine (A) nucleotide present in the female corresponding sequence; (iii) a codon coding for valine (Val) instead of phenylalanine (Phe) present in an amino acid sequence coded by the female corresponding sequence; and (iv) a codon coding for methionine (Met) instead of valine (Val) present in an amino acid sequence coded by the female corresponding sequence.

In some examples, the nucleic acid is one in which within the comparison window, there is a codon in which G is replaced with T, when said sequence is compared to the corresponding sequence in the respective region in the female FSHR gene.

In some examples, the nucleic acid is one encoding for an amino acid sequence in which within the comparison window, Phe is replaced with Val, when said sequence is compared to the corresponding sequence in the respective region in the female FSHR amino acid sequence. In some particular embodiments, the Phe which is replaced with Val is at position 587 of the FSHR amino acid sequence of M. Cephalus.

In some examples, the nucleic acid is one in which within the comparison window, there is a codon in which A is replaced with G, when said sequence is compared to the corresponding sequence in the respective region in the female FSHR gene.

In some other examples, the nucleic acid is one encoding an amino acid sequence in which within the comparison window, Val is replaced with Met, when said sequence is compared to the corresponding sequence in the respective region in the female FSHR amino acid sequence. In some particular embodiments, the Val which is replaced with Met is at position 578 of the FSHR amino acid sequence of M. Cephalus.

In some examples, the nucleic acid is one in which both above nucleic acid replacements and/or both the above amino acid replacements occur.

As noted above, a particular example of the present disclosure concerns the SD marker in M. Cephalus.

In some examples, particularly, although not exclusively, when the fish is M. Cephalus, the nucleic acid is one that corresponds to a region located between positions 1500 and 2000 of the nucleic acid sequence coding for the FSHR gene of the M. Cephalus.

In some examples, when the fish is M. Cephalus, the nucleic acid is one that corresponds to a region located between positions 1400 and 2100, or between positions 1400 and 2000, or between positions 1600 and 2000, or between positions 1700 and 2000, or between positions 1800 and 2000, or between positions 1900 and 2000, or between positions 1500 and 2100, or between positions 1600 and 2100, or between positions 1700 and 2100, or between positions 1800 and 2100, or between positions 1900 and 2100, or between positions 2000 and 2100, or between positions 1000 and 2500.

In one example, when the fish is M. Cephalus, the SD marker or the nucleic acid sequence for use according to the present disclosure corresponds to a sequence located between positions 1732 and 1875 of the coding sequence of the FSHR gene.

In one specific example, the SD marker or the nucleic acid sequence for use according to the present disclosure comprises a nucleic acid sequence as denoted by SEQ ID NO: 50, or a sequence having high homology therewith and which includes at least the SD marker disclosed herein for the M. Cephalus fish.

In one further specific example, the SD marker or the nucleic acid sequence for use according to the present disclosure is one encoding for an amino acid sequence denoted by SEQ ID NO: 12, or an amino acid sequence having high homology therewith and which includes at least the SD marker disclosed herein for the M. Cephalus fish.

In some examples, the SD marker or the nucleic acid sequence for use according to the present disclosure is one having the at least one nucleotide difference at position 1759, and/or at position 1732 of the coding sequence of the FSHR gene.

In one particular example, the at least one nucleotide difference according to the present disclosure is a SNP located at position 1759 of the coding sequence of the FSHR gene.

In some particular examples, the SD marker or the nucleic acid sequence for use according to the present disclosure comprises a sequence as denoted by SEQ ID NO: 19 or a sequence having high homology to said SEQ ID NO: 19 when optimally aligned therewith.

In some particular examples, the mRNA of the male form of the M. cephalus FSHR gene comprises a nucleic acid sequence as denoted by SEQ ID: 19.

In some further examples, the mRNA of the female form of the M. cephalus FSHR gene comprises a nucleic acid sequence as denoted by SEQ ID: 13, or SEQ ID: 15, or SEQ ID: 17.

Determination of the sex determination marker in Mugilidae fish is particularly useful for aquaculture, since it enables to define the sex of a fish at an early step i.e. before the appearance of sex phenotype.

In the context of the present disclosure, when referring to early determination or early stage, or “before the appearance of sex phenotype” it is to be understood to refer to an age of the fish before one or more sex related phenotypes appear, as known to those versed in the art, and/or at a stage at which the fish is still immature, e.g. is a larvae.

In some examples, the early stage is the period from day 1 to the age of 1 year, at times, between 1 day and one to several months. Those versed in the art would know how to determine the period of a particular fish genera at which it is considered immature, and/or how to determine the sex related phenotypes that need to be absent in order for the fish to be considered still immature.

The nucleic acid sequence disclosed herein can be used, in accordance with some aspects of the present disclosure, in a method of sex determination in an immature Mugilidae fish. The method comprises determining presence of a SD marker of a male Mugilidae fish, the SD marker comprising the nucleic acid sequence that corresponds to a sequence of a FSHR gene of the Mugilidae fish and that includes at least one male (Y) specific nucleotide difference when compared to a corresponding sequence on a FSHR gene of a female Mugilidae fish as described herein; wherein presence of said SD marker being indicative that the fish is a male Mugilidae fish.

In the context of the present disclosure, the determination of presence of the SD marker can be achieved by any technique known in the art, including without being limited thereto, PCR, DNA sequencing such as Sanger sequencing, High-throughput Sequencing or Next-generation sequencing, or in situ hybridization.

The nucleic acid sequence disclosed herein can be used, in accordance with some aspects of the present disclosure, in a method of sex inversion of an immature Mugilidae fish. Such method can utilize, in addition to the determination of the SD marker in the fish, sex steroids, to cause the sex inversion.

Sex steroids in larvae feed is often used in commercial production, to cause sex inversion in fish species. However, sex reversal is efficient only at young age prior to visible sex differences and a genetic marker for SD therefore enables to efficiently sex reverse fishes.

Thus, a further aspect of the present disclosure provides a method of sex-reversal of an immature Mugilidae fish, i.e. prior to appearance of a sex phenotype, the method comprising
(a) identifying in the fish presence or absence of a SD marker of the male Mugilidae fish, the SD marker comprising a nucleic acid sequence that corresponds to a sequence of a FSHR gene of the Mugilidae fish and includes at least one male specific nucleotide difference when compared to a corresponding sequence on a FSHR gene of a female Mugilidae fish (XX); and
(b) treating fish with a sex reversal steroid selected according to presence or absence of the Y nucleic acid sequence,

wherein when the SD marker is present, the fish is treated with a sex reversal steroid causing sex reversal of the treated fish from a male phenotype into a female phenotype (XY females); and wherein the SD marker is absent, the fish is treated with a sex reversal steroid causing sex reversal of the treated fish from a female phenotype into a male phenotype (XX males).

A “sex reversal steroid” refers to a steroid compound i.e. a biologically active organic compound which may cause sex reversion of a young fish when administered at a suitable age and in a suitable effective amount. Those versed in the art are familiar with the various available sex reversal steroids and/or how to search and locate such compounds.

In some examples, the fish is identified as having the SD marker (i.e. is a male fish), in which case the fish is treated with a male sex reversal steroid.

In some examples, the sex reversal steroid from male to female phenotype is or comprises 17β-estradiol or a functional analog thereof. The steroid 17β-estradiol (also named Estradiol E2 or Estra-1,3,5(10)-triene-3,17β-diol or 17β-Oestradiol) is an estrogen steroid hormone.

In some examples, the fish is identified as lacking the SD marker (i.e. is a female fish), in which case the fish is treated with a female sex reversal steroid. In some examples, the sex reversal steroid from male to female phenotype is or comprises 17α-methyltestosterone also named Methyltestosterone or RU-24400 or NSC-9701 or 17α-Methylandrost-4-en-17β-ol-3-one (recognized as an androgen and anabolic steroid (AAS) compound).

In the context of the present disclosure the fish can also be treated with functional analogs of the above identified sex reversal hormone. When referring to functional analogs it is to be understood to encompass compounds that can at least cause the same sex reversal as caused by 17β-estradiol (from male to female phenotype) or 17α-methyltestosterone (from female to male phenotype).

The sex reversal hormones may be administered to fishes by any relevant administration modes. In some examples, the fish are given the hormone by oral administration, e.g. by feeding. In some additional or alternative examples, the fish are given the hormone by parenteral administration.

In some examples, the treatment of the fish, for achieving the desired sex reversal, comprises oral administration (feeding) with a dose within the range from 1 mg/kg and 500 mg/kg, at times, between 10 mg/kg and 100 mg/kg, at times, between 100 mg/kg and 500 mg/kg of food.

In some specific examples, when the fish is identified as having the SD marker, i.e. is identified as an immature male fish, the oral administration dose of the sex reversal hormone (from male to female) is between 100 to 200 mg/kg of food.

In some specific examples, when the fish is identified as lacking the SD marker, i.e. is identified as an immature female fish, the oral administration dose of the sex reversal hormone is between 100 to 200 mg/kg of food.

In some particular examples, the sex reversal steroid is 17β-estradiol and is orally administered at a dose between 100-200 mg/kg of food.

In some alternative examples, the sex reversal steroid is 17α-methyltestosterone and is orally administered at a dose between 10-20 mg/kg of food.

As noted above, sex reversal has an advantage when conducted at young age, i.e. when the fish is immature and prior to visible sex differences. The present disclosure allows the use of the newly identified genetic marker for SD.

In some aspects, the newly identified SD marker can be used for creating a mono-sex population of fish.

In some examples, a female mono-sex population can be constructed by female-to-male sex reversal, followed by mating of the XX males (after sex reversal) with females. The most reliable marker for sex would be the causal variation in the MKR of SD.

Therefore, in a further aspect, the present disclosure provides a method for producing a mono-sex population of female Mugilidae fishes, the method comprising:

(a) providing a Mugilidae fish at an age prior to appearance of a sex phenotype;

(b) identifying in said fish presence or absence of a sex determination (SD) marker of a male Mugilidae fish, the SD marker comprising a nucleic acid sequence that corresponds to a sequence of a FSHR gene of the Mugilidae fish and includes at least one Y specific nucleotide difference when compared to a corresponding sequence on a FSHR gene of a female Mugilidae fish (XX);

(c) selecting fish that is absent of said SD marker (i.e. is an immature female fish);

(d) treated the selected fish with an amount of a male sex steroid, the amount being effective to cause sex reversal of the fish that is absent of the SD marker, into a reversed male fish; and

(c) allowing said reversed male fish (XX) to mate with a native female fish (XX), to thereby obtain a mono-sex population of female fishes.

In some examples of this method of producing a mono-sex population, the sex steroid is 17α methyltestosterone or a functional analog thereof. The administration modes and doses may be as previously detailed above.

Accordingly, the present disclosure, provides, for the first time, a method to obtain a mono-sex population of Mugilidae fishes comprising exclusively immature female fishes, i.e. a population of a selected gender before the appearance of the sex phenotype.

Thus, in a further aspect, the present disclosure provides a mono-sex population of immature female Mugilidae fishes, i.e. wherein said fishes are at an age before appearance of a sex phenotype.

The mono-sex population of the present disclosure can be characterized by unique genetic features, including, the absence of the nucleic acid sequence identified herein as the male SD marker.

In some specific examples, the fish of the mono-sex population is a female M. Cephalus and the nucleic acid sequence comprises G at position 1732 and A at position 1875 of the coding sequence of the FSHR gene.

In some examples, the mono-sex population of the present disclosure is obtained by performing the above described method.

The mono-sex population can be contained/maintained in a fish cultivating reservoir. The reservoir can be in the form of a pond, cultivating tank etc. Thus, in accordance with some further aspects, the present disclosure also provides a fish cultivating reservoir comprising a mono-sex population of Mugilidae immature fishes.

The identification of a SD marker in an organism of interest enables also to manipulate genomic region using suitable genetic tools commonly known in the art in order to reverse the sex of the organism.

Thus, another aspect of the present invention relates to a genetic manipulation method of sex-reversal of a Mugilidae fish, comprising introducing in a fish embryo a gene-editing agent for modifying a sex determination marker of said fish, wherein the sex determination marker comprises a nucleic acid sequence corresponding to a sequence of an FSHR gene of said fish and including at least one male specific nucleotide difference when compared to a corresponding sequence on an FSHR gene of a female Mugilidae fish.

In some examples, the genetic manipulation method can make use of gene editing agents. For example, and without being limited thereto, the gene editing agents can be any one of a Clustered Regularly Interspersed Short Palindromic Repeats (CRISPR)/Cas system agent, a Transcription Activator-Like Effector Nuclease (TALEN) agent, a Zinc-Finger Nucleases (ZFN) agent or an anti-sense RNA agent.

As used in the specification and claims, the forms “a”, “an” and “the” include singular as well as plural references unless the context clearly dictates otherwise. For example, the term “a fish” includes one or more fishes and the term “fishes” includes one fish as well as more than one fish.

As used herein, the term “or” means one or a combination of two or more of the listed choices.

Further, as used herein, the term “comprising” or “including” is intended to mean that the nucleic acid sequences, methods and populations includes the recited elements, but does not exclude others. Similarly, “consisting essentially of” is used to define nucleic acid sequences, methods and populations that include the recited elements but exclude other elements that may have an essential significance on the functionality of the nucleic acid sequences, methods and populations of the inventions. “Consisting of” shall mean excluding other elements. Embodiments defined by each of these transition terms are within the scope of this invention.

Further, all numerical values, e.g., dose or ranges thereof, are approximations which are varied (+) or (−) by up to 20%, at times by up to 10%, from the stated values. It is to be understood, even if not always explicitly stated that all numerical designations are preceded by the term “about”. It also is to be understood, although not always explicitly stated, that the reagents described herein are merely exemplary and that equivalents of such are known in the art.

### NON-LIMITING EXAMPLES

### Experimental Procedures

### Fish Sampling and Sexing

Adult flathead grey mullet specimens from two full sib families (n=6 and n=7) and from a wild population (n=70) were dissected and sexed based on external observation of gonad morphology (n=83). Additionally, the gonads of 27 individuals (17 males and 10 females), were also examined by microscope in order to improve confidence of the visual sexing. A caudal fin sample was taken from each of the adult specimens and kept in ethanol (90-96%). Fin samples were also taken from two groups of 28 days post hatch larvae (Group A, n=7; Group B, n=8, Dor et al., 2020), and from 86 larvae of a wild population.

Sequence Assembly of FSHR in M. cephalus

M. cephalus FSHR raw reads (accession number PRJEB12265) and previously built SOAPdenovo scaffolds [2] of a male and female were separately assembled. The Gap5 (Staden R, et al, (2000) Bioinformatics Methods and Protocols, Humana Press: New Jersey, pp 115-130) wrapper and the BWA program was used for alignment of reads and scaffolds against the masked gene sequence of Dicentrarchus labrax Fshr (GenBank accession no. FQ310507). Masking was performed using CENSOR (Kohany O, et al (2006) BMC Bioinformatics 7: 474) and Tilapia Repeat Masker version 2 (Shirak A, et al. (2010) Mar Biotechnol 12: 121-125). Aligned sequences were then used for de novo assembly using Gap4 (Staden R, et al, (2000) Bioinformatics Methods and Protocols, Humana Press: New Jersey, pp 115-130). Raw reads and scaffolds were used again for alignment against the new assembled sequences using Gap5. A consensus sequence was obtained and used for further alignment. This process was iterated few times until the entire gene assembly was completed. Exon borders were deduced using mRNA sequences using the Trinity software.

### DNA Extraction and Sequencing Procedure

A sample of the caudal fin (100-200 mg) was used for DNA extraction using a commercial kit (MasterPure DNA Purification, Madison, USA). Polymerase Chain Reaction (PCR) was performed using relevant primers (Table 1) using the Bio-X-ACT™ Long kit (Bioline Ltd., London, UK) according to the manufacturer's instructions under the following conditions: 36 cycles for 30 s at 94° C., 30 s at 60° C. and 30 s at 72° C. Following, the PCR products were excised from the gel and purified with a kit (Montage Gel Extraction, Millipore, Bedford, Mass., USA). Sanger sequencing was conducted from both directions of the amplified genomic region. For the purpose of allele specific PCR, “X” specific and “Y” specific forward primers were designed following the method for allele specific PCR of Liu et al. (Liu J, et al. (2012) Plant Methods 8: 34), including a mismatch in three nucleotides at the 3′ end. Annealing temperature was raised to 69° C.

TABLE 1
PCR primer pairs.

| | | Product | | SEQ |
|---|---|---|---|---|
| | | size | Target of | ID |
| Pair | Sequence (5′ to 3′) | (bp) | amplification¹ | NO. |
| 1 | GGATGTGGAGGATCTGGTGT | 221 | SD region (Exon | 1 |
| | GAGACGGCGAAGAAGGAGAT | | 14) | 2 |
| 2 | CGGAATCTAAGGTCCTCCTG | 491/481 | Upstream of SD | 3 |
| | GCCTTCTTCTTTGCCTGAGA | | region (Exon 14) | 4 |
| 3 | GCCGGCCCACGCCGACACGCTCG | 618/608 | FSHRY | 5 |
| | GCCTTCTTCTTTGCCTGAGA | | | 6 |
| 4 | GCCGGCCCACGCCGACACGCTCA | 618/608 | FSHRX | 7 |
| | GCCTTCTTCTTTGCCTGAGA | | | 8 |
| 5 | CGGTGACGATACTGGTGATG | 230 | Nonsynonymous | 9 |
| | CTTCAATGGCTGCAGGACAC | | SNP (Exon 1) | 10 |

¹FSHRX represents the putative non-specific allele of FSHR, while FSHRY represents the male-specific allele.

### Characterization of Sex-Specific Alleles for FSHR

Analysis of genomic alleles was based on deep sequencing of a male and female deposited in the Short Read Archive (SRA) under project IDs PRJEB12265 and PRJEB34342, respectively. Based on sequence variation along the assembled FSHR gene and read-pair information, sex-specific alleles were manually separated using GAPS viewer.

### FSHR Gene Expression

Expression previously established RNA-seq libraries included two groups of larvae A and B (n=7 and 8), Female and male brains (n=3 and 2, respectively), female and male gonads (n=2 and 1, respectively). Existence of FSHR variants e.g., male-specific allele of FSHR (FSHRY), and the non-specific allele of FSHR (FSHRX), were examined in these libraries, using the BLASTN algorithm for alignment. FSHR expression values were calculated and normalized in a previous study using Trimmed Mean of values (TMM).

### Statistics

The JMP® statistical package (Pro 13, SAS Institute, Cary, N.C.) was used for conducting statistical tests. Fisher's exact Chi-squared test was applied for association study of individual genetic markers and sex. Pearson Chi-squared test was applied for testing deviation from Hardy-Weinberg equilibrium.

### Example 1

Assembly of FSHR in M. cephalus

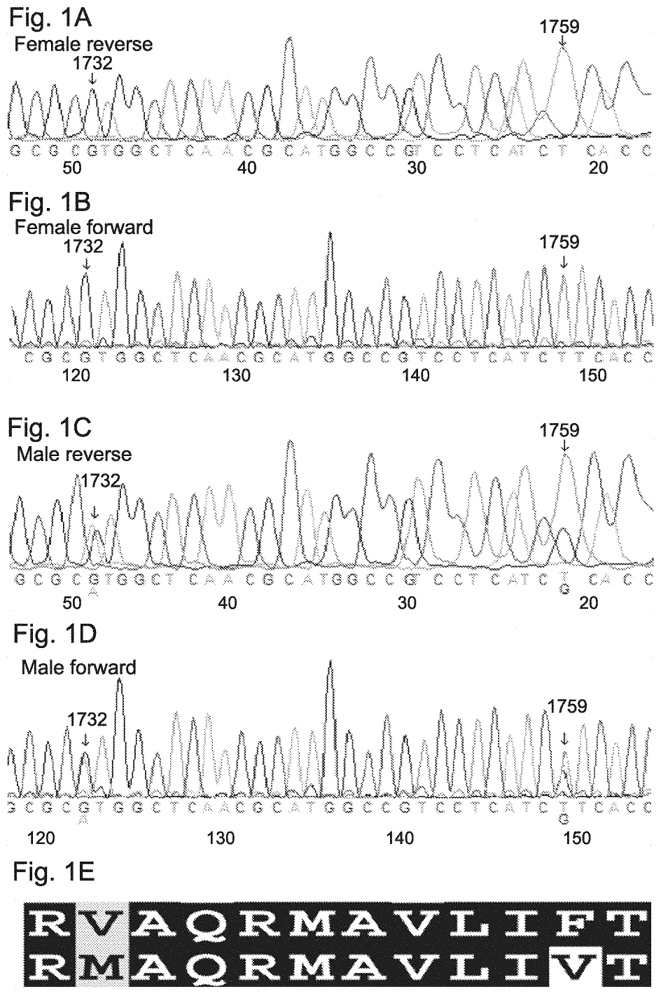

The established genomic libraries allowed the assembly of the whole FSHR gene in two individuals of M. cephalus representing both sexes. Separation of sequencing fragments based on polymorphism in their paired-reads sequences revealed four different FSHR alleles, with no evidence for a fifth allele that could indicate a copy-number variation (CNV). Annotation based on the RNA-seq data was used to characterize the FSHR gene structure with its 14 exons, its conserved exon-intron boundaries and its putative protein sequence, which were found similar to those of known orthologous FSHRs (Table 2,). Using this predicted FSHR protein and the BLASTP program; the reference sequences deposited in GenBank of mammals were searched, for amphibians, birds and reptilians. This search corroborated that the gene is FSHR, e.g. 50% identity and 67% similarity to human FSHR, NP_000136 (as denoted by SEQ ID NO: 55); 51% and 66% similarity to clawed frog FSHR, XP_017949625 (as denoted by SEQ ID NO: 71); 50% identity and 66% similarity to duck FSHR, XP_005012152 (as denoted by SEQ ID NO: 72); and 51% identity and 65% similarity to python FSHR, XP_007431488 (as denoted by SEQ ID NO: 52). Yet, nomenclature for many similar proteins in other fishes was different e.g. 77% identity and 84% similarity to Nile tilapia gonadotropin receptor I (gth-ri), NP_001266517 (as denoted by SEQ ID NO: 57); and 49% identity and 64% similarity to Fugu lutropin-choriogonadotropic hormone receptor, XP_029691459 (as denoted by SEQ ID NO: 61). However, synteny analysis indicated that in spite of the different nomenclatures all these similarities arise from the same ancestral gene, e.g. Nile tilapia gth-ri that is located on LG8 in position orthologous to mullet FSHR on LG9 [2]. The mullet's exonic FSHR SNPs that were capable of encoding amino acid changes were marked for analysis, and sex-biased polymorphism was used to assemble the male specific (one allele) and nonspecific sequences of FSHR (three allele), representing the “Y” and “X” genomic regions, designated as FSHRY and FSHRX, respectively (Table 3 and FIG. 1).

The nucleic acid sequence of the Mugil cephalus follicle-stimulating hormone receptor, male form (FSHRY), complete gene and flank is as denoted by SEQ ID NO: 11. The nucleic acid sequence of the Mugil cephalus follicle-stimulating hormone receptor, female form (FSHRX) Female_A1 mRNA is as denoted by SEQ ID NO: 13, encoding for the amino acid sequence as denoted by SEQ ID NO: 14. The nucleic acid sequence of the Mugil cephalus follicle-stimulating hormone receptor, female form (FSHRX) Female_A2 mRNA is as denoted by SEQ ID NO: 15, encoding for the amino acid sequence as denoted by SEQ ID NO: 16. The nucleic acid sequence of the Mugil cephalus follicle-stimulating hormone receptor, female form (FSHRX) Male_x mRNA is as denoted by SEQ ID NO: 17, encoding for the amino acid sequence as denoted by SEQ ID NO: 18. The nucleic acid sequence of the Mugil cephalus follicle-stimulating hormone receptor, male form (FSHRY) Male_Y mRNA is as denoted by SEQ ID NO: 19, encoding for the amino acid sequence as denoted by SEQ ID NO: 20.

TABLE 2
Genomic organization of the Mugil cephalus FSHR gene based on the
male FSHRY variant.

| SEQ | | | | SEQ | | |
|---|---|---|---|---|---|---|
| ID | | Exon | | ID | | |
| NO: | Intron¹ | no. | size | NO: | Intron | size |
| 21 | [symbol] ATG | 1 | 188 | 22 | GTGCTTgtgagtacgc | 3119 |
| 23 | catccacagGGAACT | 2 | 72 | 24 | GAGACTgtaagcttta | 120 |
| 25 | caactttagGAACAT | 3 | 75 | 26 | TTACATgtgagtgtgt | 526 |
| 27 | ctgccccagCTTAAT | 4 | 75 | 28 | TGAGATgtaagtataa | 107 |
| 29 | tcctttcagAACCAT | 5 | 75 | 30 | GTTTATgtgagtactc | 885 |
| 31 | togtcacagGACCAT | 6 | 75 | 32 | TCAGCTgtgagtctca | 319 |
| 33 | gacactcagTCACCT | 7 | 81 | 34 | GAAGATgtaagggctt | 1506 |
| 35 | tattgacagATGGCT | 8 | 69 | 36 | AAGACTgtgagtcaca | 101 |
| 37 | tccccttagATTTCT | 9 | 75 | 38 | AGTGCTgtaagcatga | 380 |
| 39 | ctcccacagTGACAT | 10 | 186 | 40 | AAACAAgtgcgcgtct | 115 |
| 41 | gtgtgctagATCAAG | 11 | 218 | 42 | TGTTGAgtatgaaatg | 140 |
| 43 | cattgtcagGCAGCA | 12 | 182 | 44 | TTCTCGgtgtgtacgc | 97 |
| 45 | cacgtgcagGTGTTT | 13 | 192 | 46 | AGCAAAgtgggtgtta | 1046 |

| | | | | | |
|---|---|---|---|---|---|
| 47 | ttcgtgcagGTGAGC | 14 | 529 | 48 | CGTGCG [symbol] |

¹Intron and exon sizes are given in base pairs, and their sequences are written in lowercase and uppercase letters, respectively. The first and last two bases of the introns are in bold type (gt and ag for donor and acceptor splice sites, respectively). The initiation and stop codons are in bold and underlined (ATG, TAG). Starting from the initiation codon, the genomic and putative transcript sizes of the FSHRY gene are 10,553 and 2,118 bp, respectively.

### Example 2

### Analysis of Polymorphism in FSHR

Two nonsynonymous male-specific SNPs were initially found in Exon 14 at 1732 and 1759 nucleotide positions of FSHR assembled sequence (Table 3). Using PCR primers (pair 1, Table 1), these SNPs were investigated in two full sib families (n=6 and n=7), and were found to be fully associated with sex. Another two nonsynonymous SNPs in exons 1 and 14 (Table 3, nucleotide positions at 131 and 2047 bp, respectively) were found in FSHR's assembly; however, they did not fit the expected XY/XX SD model, as both SNPs were homozygous for each variant in the two established genomic libraries for both sexes that were used for assembly. Using PCR primers (pairs 2 and 5, Table 1), the relevant regions in exons 1 and 14 were amplified and sequenced in a small panel of WT males (n=4) and females (n=4), negating their association with sex. Moreover, they did not fit any simple genetic sex chromosome model (XY/XX or WZ/ZZ), as homozygous individuals were found for each variant. Except for these incidences, Sanger sequencing revealed another two nonsynonymous SNPs at nucleotide position 149 and 1781 bp, in exons 1 and 14, respectively (Table 3), but they were not associated with sex.

TABLE 3
Polymorphism in M. cephalus predicted FSHR gene.

| | Exon 1 | | Exon 14¹ | | | | | | | | |
|---|---|---|---|---|---|---|---|---|---|---|---|
| Position² | 131 | 149 | 1689 | 1732 | 1759 | 1781 | 1875 | 1917 | 2031 | 2047 | 2112 |
| Nucleotide | T/C | T/C | C/A | G/A | T/G | C/A | C/T | C/T | G/A | G/A | C/A |
| AA | L/P | M/T | T/T | V/M | F/V | A/D | I/I | R/R | S/S | A/T | S/S |

¹Two non-synonymous SNPs comprising a haplotype that fits an XY model of SD are in bold and underlined font. The “Y” FSHRY haplotype is presented on the right side of the divider.
²Nucleotide position in the coding DNA sequence (in bp). Nucleotide and amino acid (AA) variation is denoted in both sides of divider.

### Example 3

### Two Male-Specific Non-Synonymous Substitutions in FSHR are Male-Specific

The two male-specific SNPs (c.1732G>A and c.1759T>G representing FSHRY) found in the full sib families, which are capable of encoding non-conservative amino acid substitutions (Table 3, FIG. 1), were further investigated for association with sex in WT samples. All tested samples (n=83) showed these two SNPs were a stable haplotype which is highly significantly associated with sex with only 4.8% of mismatches (p≤6.7×10⁻¹⁹; Table 4). Samples showing discordance between FSHRY haplotype and sex were all phenotyped for sex without microscope inspection. In addition, none of these samples (n=83) nor other non-phenotyped samples (n=86), were homozygous for the FSHRY haplotype, even though their expected frequency based on Hardy-Weinberg equilibrium was 6.6% (Table 5). Most importantly, on the population level, additional SNPs that were flanking the FSHRY haplotype, including other non-synonymous substitutions (Table 3, positions 131-1689 and 1917-2112), did not show significant association with sex, and thus delimit the SD associated region, between positions 1732 and 1875, to 143 nucleotides on Exon 14 (Table 3), corresponding to the nucleic acid sequence as denoted by SEQ ID NO: 50 and encoding for the amino acid sequence as denoted by SEQ ID NO: 12.

TABLE 4
Association between FSHR haplotype for SNPs at nt 1732
and 1759 on exon 14, and phenotypic sex of M. cephalus.
The homozygous haplotype represents “XX”, while the heterozygous
haplotype represents “XY”¹.

| FSHR haplotype | Female | Male | Total count |
|---|---|---|---|
| “XX” | 37 | 1 | 38 |
| “XY” | 3 | 42 | 45 |
| Total count | 40 | 43 | 83 |

¹Fisher exact test: p < 6.7 × 10⁻¹⁹.

TABLE 5
Contingency table comparing observed counts and frequencies
of FSHR haplotypes with those expected by Hardy-Weinberg
equilibrium in the genotyped population (N = 170) ¹.

| | Actual count | Expected count |
|---|---|---|
| FSHR haplotype | (frequency) | (frequency) |
| “XX” | 83 (0.48) | 94 (0.55) |
| “XY” | 87 (0.52) | 65 (0.38) |
| “YY” | 0 (0) | 12 (0.07) |

¹ Pearson Chi-square test for divergence of FSHR haplotypes from Hardy-Weinberg equilibrium (p ≤ 3.9 × 10⁻⁵).

### Example 4

### Conservation of FSHR Protein Among Vertebrate Species

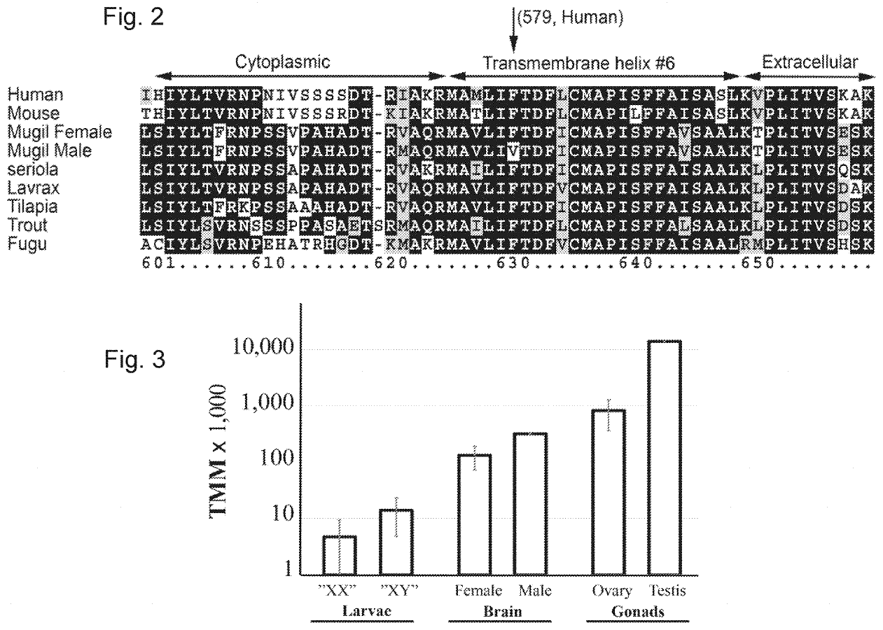

The protein sequence of the FSHRY variant was used as a template for BLASTP search against GenBank (non-redundant protein sequences). While the V/M substitution (nt 1732 on exon 14) was found in other organisms, the F/V substitution (nt 1759 on exon 14) is novel and alters an amino acid, which is conserved across vertebrate species (FIGS. 1 and 2).

### Example 5

### Analysis of Full Sib-Groups for Sex-Biased Expression

Two full sib groups of fry (28 days post hatch (dph)) of a mullet family (n=15), have been assigned to different sexes, designated as A and B, with differential expression of genes that are located on the critical SD chromosomal region. FSHR was genotyped for these groups and it revealed that they represent FSHR haplotypes “XX” and “XY”, respectively (Table 5). Interestingly, FSHRY was not found in any expression library, whereas expression of FSHRX varied between larvae, adult brains and gonads, with tendency towards higher values in male tissues (FIG. 3).

1-50. (canceled)

51. A sex determination (SD) marker of a male Mugilidae fish comprising a nucleic acid sequence corresponding to a sequence of a Follicle-stimulating hormone receptor (FSHR) gene of said fish and including at least one male (Y) specific nucleotide difference when compared to a corresponding sequence on a FSHR gene of a female Mugilidae fish.

52. The SD marker of claim 51, wherein at least one of:
(a) said SD marker corresponds to a sequence encoding a G protein interaction domain loop of the FSHR protein;
(b) said SD marker corresponds to a sequence encoding at least one of transmembrane α-helices six (TM6) domain and intracellular loop 3 (IL3) domain of FSHR protein;
(c) each of the at least one male (Y) specific nucleotide difference is a Single-Nucleotide Polymorphism (SNP);
(d) wherein each of the at least one nucleotide difference is located at a first or at a second nucleotide position within a codon of the SD marker.

53. The SD marker of claim 51, comprising two male specific nucleotide differences, when compared to the corresponding sequence of the FSHR gene of a female Mugilidae fish, optionally wherein said two male specific (Y) nucleotide differences are within a region of less than 150 base pairs (bp).

54. The SD marker of claim 51, corresponding to a sequence from Exon 14 of the FSHR gene, and optionally comprising within a region of less than 150 bp, at least one of (i) a Thymine (T) nucleotide instead of a Guanine (G) nucleotide present in the female corresponding sequence (ii) a Guanine (G) nucleotide instead of an Adenosine (A) nucleotide present in the female corresponding sequence; (iii) a codon coding for valine (Val) instead of phenylalanine (Phe) present in an amino acid sequence coded by the female corresponding sequence; and (iv) a codon coding for methionine (Met) instead of valine (Val) present in an amino acid sequence coded by the female corresponding sequence.

55. The SD marker of claim 51, wherein said at least one nucleotide difference comprises T in the nucleic acid sequence instead of G present in the female (X) corresponding sequence.

56. The SD marker of claim 51, wherein said Mugilidae fish is Mugil Cephalus, and wherein at least one of:
(a) the SD marker corresponds to a sequence located between positions 1500 and 2000 of the nucleic acid sequence coding for the FSHR gene of the Mugil Cephalus,
(b) the SD marker corresponds to a sequence located between positions 1732 and 1875 of the coding sequence of the FSHR gene of the Mugil Cephalus,
(c) said at least one nucleotide difference is located at position 1759, or at position 1732 or at both position 1759 and 1732 of the coding sequence of said FSHR gene,
(d) the SD marker comprises a sequence as denoted by SEQ ID NO: 19 or a sequence having at least 80% homology to said SEQ ID NO: 19 when optimally aligned therewith,
(e) the SD marker comprises a sequence encoding for an amino acid sequence as denoted by SEQ ID NO: 20 or an amino acid sequence having at least 80% homology to said SEQ ID NO: 20 when optimally aligned with it.

57. A method of sex determination in a Mugilidae fish before appearance of a sex phenotype, the method comprises determining presence of a sex determination (SD) marker of a male Mugilidae fish, the SD marker comprising a nucleic acid sequence that corresponds to a sequence of a FSHR gene of the Mugilidae fish and includes at least one male (Y) specific nucleotide difference when compared to a corresponding sequence on a FSHR gene of a female Mugilidae fish (XX);
wherein presence of said SD marker being indicative that the fish is a male Mugilidae fish.

58. The method of claim 57, wherein at least one of:
(a) said SD marker corresponds to a sequence encoding a G protein interaction domain loop of the FSHR protein;
(b) said SD marker corresponds to a sequence encoding at least one of transmembrane α-helices six (TM6) domain and intracellular loop 3 (IL3) domain of FSHR protein;
(c) each of the at least one male (Y) specific nucleotide difference is a Single-Nucleotide Polymorphism (SNP);
(d) wherein each of the at least one nucleotide difference is located at a first or at a second nucleotide position within a codon of the SD marker.

59. A method of sex-reversal of a Mugilidae fish prior to appearance of a sex phenotype, the method comprising
(a) identifying in the fish presence or absence of a sex determination (SD) marker of the male Mugilidae fish, the SD marker comprising a nucleic acid sequence that corresponds to a sequence of a FSHR gene of the Mugilidae fish and includes at least one male (Y) specific nucleotide difference when compared to a corresponding sequence on a FSHR gene of a female Mugilidae fish (XX); and
(b) treating fish with a sex reversal steroid selected according to presence or absence of said nucleic acid sequence,
wherein when said SD marker is present, the fish is treated with a sex reversal steroid causing sex reversal of the treated fish from a male phenotype into a female phenotype; and
wherein said SD marker is absent, the fish is treated with a sex reversal steroid causing sex reversal of the treated fish from a female phenotype into a male phenotype.

60. The method of claim 59, wherein at least one of:
(a) said SD marker corresponds to a sequence encoding a G protein interaction domain loop of the FSHR protein;
(b) said SD marker corresponds to a sequence encoding at least one of transmembrane α-helices six (TM6) domain and intracellular loop 3 (IL3) domain of FSHR protein;
(c) each of the at least one male (Y) specific nucleotide difference is a Single-Nucleotide Polymorphism (SNP);
(d) wherein each of the at least one nucleotide difference is located at a first or at a second nucleotide position within a codon of the SD marker.

61. The method of claim 59, wherein said fish is identified as having the SD marker and said sex reversal steroid is 17β-estradiol or a functional analog thereof, optionally wherein said method comprises treating the fish with an oral dose of between 100 to 200 mg/kg of food.

62. The method of claim 59, wherein said fish is identified as lacking the SD marker and said sex reversal steroid is 17α-methyltestosterone or a functional analog thereof, optionally wherein said method comprises treating the fish with an oral dose of between 10 to 20 mg/kg of food.

63. The method of claim 59, wherein treating comprises oral administration of the sex reversal hormone.

64. A method for producing a mono-sex population of female Mugilidae fishes, the method comprising:
(a) providing a Mugilidae fish at an age prior to appearance of a sex phenotype;
(b) identifying in said fish presence or absence of a sex determination (SD) marker of a male Mugilidae fish, the SD marker comprising a nucleic acid sequence that corresponds to a sequence of a FSHR gene of the Mugilidae fish and includes at least one male (Y) specific nucleotide difference when compared to a corresponding sequence on a FSHR gene of a female Mugilidae fish (XX);
(c) selecting fish that is absent of said SD marker;
(d) treated the selected fish with an amount of a male sex steroid, the amount being effective to cause sex reversal of the fish into a reversed male fish (XX); and
(c) allowing said reversed male fish (XX) to mate with a female fish (XX), to thereby obtain a mono-sex population of female fishes.

65. The method of claim 64, wherein at least one of:
(a) said SD marker corresponds to a sequence encoding a G protein interaction domain loop of the FSHR protein;
(b) said SD marker corresponds to a sequence encoding at least one of transmembrane α-helices six (TM6) domain and intracellular loop 3 (IL3) domain of FSHR protein;
(c) each of the at least one male (Y) specific nucleotide difference is a Single-Nucleotide Polymorphism (SNP);
(d) wherein each of the at least one nucleotide difference is located at a first or at a second nucleotide position within a codon of the SD marker.

66. The method of claim 64, wherein said male sex steroid is 17α methyltestosterone or a functional analog thereof.

67. A mono-sex population of Mugilidae fishes, wherein said fishes are at an age before appearance of a sex phenotype.

68. The mono-sex population of claim 67, wherein each fish member of the population does not comprise a nucleic acid sequence corresponding to a sequence of a FSHR gene of the Mugilidae fish and including at least one male specific nucleotide difference when compared to a corresponding sequence of a FSHR gene of a female Mugilidae fish, optionally wherein the fish is M. Cephalus and the nucleic acid sequence comprises Guanine at position 1732 and Adenosine at position 1875 of the coding sequence of the FSHR gene.

69. A fish cultivating reservoir comprising a mono-sex population of claim 68.

70. A method of sex-reversal of a Mugilidae fish, comprising introducing in a fish embryo a gene-editing agent for modifying a sex determination marker of said fish, wherein said sex determination marker comprises a nucleic acid sequence corresponding to a sequence of an FSHR gene of said fish and including at least one male (Y) specific nucleotide difference when compared to a corresponding sequence on an FSHR gene of a female Mugilidae fish.
